(19) United States
(12) Patent Application Publication
(10) Pub. No.: US 2015/0011416 A1
(43) Pub. Date: Jan. 8, 2015

(54) MULTIMODAL PCR TARGET DETECTION

(71) Applicant: Qiagen Mansfield, Inc, Mansfield, MA (US)

(72) Inventors: Jason Lei, Brentwood, TN (US); Lilly l. Kong, Covina, CA (US); Kiran Madanahally Divakar, Shrewsbury, MA (US)

(21) Appl. No.: 14/379,615

(22) PCT Filed: Feb. 22, 2013
(86) PCT No.: PCT/US2013/027383

§ 371 Date: Aug. 19, 2014

Related U.S. Application Data
(60) Provisional application No. 61/602,244, filed on Feb. 23, 2012.
(60) Provisional application No. 61/602,246, filed on Feb. 23, 2012.
(60) Provisional application No. 61/671,314, filed on Jul. 13, 2012.
(60) Provisional application No. 61/671,315, filed on Jul. 13, 2012.

(51) Int. Cl. C12Q 1/68

(52) U.S. Cl. CPC C12Q 1/686
USPC 506/9; 435/6.12; 435/6.11

(57) ABSTRACT

The technology described herein relates to detecting the presence of one or more specific nucleic acids, e.g. determining the genotype of one or more allelic target site loci.

### CROSS-REFERENCE TO RELATED APPLICATIONS

This application claims benefit under 35 U.S.C. §119(e) of U.S. Provisional Application Nos. 61/602,246 and 61/602,244 filed Feb. 23, 2012 and 61/671,315 and 61/671,314 filed Jul. 13, 2012, the contents of each of which are incorporated herein by reference in their entirety.

### SEQUENCE LISTING

The instant application contains a Sequence Listing which has been submitted in ASCII format via EFS-Web and is hereby incorporated by reference in its entirety. Said ASCII copy, created on Feb. 22, 2013, is named 046264-072554-PCT_SL.txt and is 14,473,728 bytes in size.

### FIELD OF THE INVENTION

The claimed invention relates to methods of detecting the presence of one or more specific nucleic acids. In some embodiments, the methods determine the genotype of one or more allelic target site loci. In some embodiments, the methods relate to multiplex PCR.

### BACKGROUND

Current PCR-based methods for detecting the presence of single nucleotide polymorphisms (SNPs) and/or mutations in a sample suffer from background signal. For example, one common approach for detection of SNPs is Amplification Refractory Mutation System (ARMS). The allele-specific primers utilized in ARMS are designed to comprise only target-specific sequences with variation at the 3′ terminal nucleotide such that only the primer whose 3′ terminal nucleotide is complementary to a given template can be extended, thereby identifying the nucleotide at the SNP site. Such primers can misprime due to non-specific interactions with non-target sequences, i.e. the ARMS approach is “leaky.” Additionally, when ARMS is multiplexed and at least one amplicon accumulates to high levels, primer interaction or “crosstalk” occurs.

Reactions utilizing nested primers have been attempted. In these approaches, a first set of non-allele specific primers is used to amplify all alleles of a target locus present in a sample, using a first annealing temperature. A second set of allele-specific primers is then used to amplify the target at a second, lower annealing temperature. This approach can suffer from the same issues of leakiness and crosstalk as described for the ARMS approach (Tabone et al. BMC Genomics 2009 10:580).

### SUMMARY OF THE INVENTION

The methods and compositions of the invention described herein permit the determination of the presence of one or more target polynucleotides in a nucleic acid sample. Various aspects of the invention are based upon the use of at least one dual domain primer (or truncated dual domain primer) in a PCR amplification regimen having at least two phases which are differentiated by having different annealing temperatures. During the first phase of the PCR amplification regimen, a 3′ core region of the dual domain primer (or truncated dual domain primer) specifically anneals to its target and primer extension occurs, creating amplification products comprising the target polynucleotide and/or its complement. These amplification products will contain “tag” sequences at at least one end of the product, the tags comprising the sequence of a 5′ tail region of the at least one dual domain primer (or truncated dual domain primer). In the second phase of the PCR amplification regimen, the annealing temperature is raised, such that sequence complementary to both the 5′ tail region and 3′ core region is required for annealing of the dual domain primer (or truncated dual domain primer) to a target, thus preventing the primer from mispriming to off-target polynucleotides.

The methods and compositions described herein can be used to reduce amplification of off-target polynucleotides and to improve the signal-to-noise ratio of a PCR amplification regimen where such parameters are of concern. In some embodiments, the methods and compositions described herein can be used to reduce amplification of off-target polynucleotides and to improve the signal-to-noise ratio of a PCR amplification regimen designed to detect alleles of one or more SNPs.

One aspect of the invention described herein relates to a method for determining the presence of one or more target polynucleotides, the method comprising; performing a PCR amplification regimen comprising cycles of strand separation, primer annealing, and primer extension on a reaction mixture comprising a nucleic acid sample and a set of oligonucleotide primers specific for each target polynucleotide; wherein each set of oligonucleotide primers comprises a first subset of at least one truncated dual domain forward primer and a second subset of at least one reverse primer; wherein each truncated dual domain primer of a set comprises a 5′ tail region that differs from the 5′ tail region on other truncated dual domain primers in the set and a 3′ core region complementary to a sequence on one strand of a double-stranded nucleic acid comprising the target; wherein for each truncated dual domain primer, the 3′ core substantially anneals to its complementary target site sequence at a first annealing temperature, and the sequence comprised by the 5′ tail and 3′ core region substantially anneals to its complement at a second annealing temperature, the second annealing temperature being higher than the first annealing temperature, such that at the second annealing temperature the 3′ core of the truncated dual domain primer cannot substantially anneal to a template molecule that does not also have the complement of the truncated dual domain primer's 5′ tail sequence; wherein for each truncated dual domain primer of a primer set: the 5′ tail sequence does not have any homology to the target sequence; and the 5′ tail sequence has 6 or fewer contiguous homologous bases relative to any other 5′ tail sequence of the truncated dual domain primer set; wherein the PCR amplification regimen comprises first and second phases, the first phase comprising annealing at the first annealing temperature for a first set of cycles, and; the second phase comprising annealing at the second annealing temperature for a second set of cycles; and detecting an amplified product for each target polynucleotide, wherein the detecting indicates the presence of the target polynucleotide.

In some embodiments, a reverse primer comprises a dual domain primer. In some embodiments, a reverse primer comprises a truncated dual domain primer. In some embodiments, a reverse primer comprises an amplifying primer.

In some embodiments, the set of primers comprises at least one dual domain forward primer; wherein each dual domain primer of a set comprises a 5′ tail region that differs from the 5′ tail region of other dual domain primers in the set, a 3′ core region complementary to a sequence on one strand of a double-stranded nucleic acid comprising said target, and a terminal nucleotide complementary to one of the variant nucleotides occurring at said target site; wherein for each dual domain primer, the 3′ core substantially anneals to its complementary target site sequence at a first annealing temperature, and the sequence comprised by the 5′ tail and 3′ core region substantially anneals to its complement at a second annealing temperature, the second annealing temperature being higher than the first annealing temperature, such that at said second annealing temperature said 3′ core of said dual domain primer cannot substantially anneal to a template molecule that does not also have the complement of the dual domain primer's 5′ tail sequence; and wherein for each truncated dual domain primer of a primer set: the 5′ tail sequence does not have any homology to the target sequence; and the 5′ tail sequence has 6 or fewer contiguous homologous bases relative to any other 5′ tail sequence of the dual domain or truncated dual domain primer set.

In some embodiments, the target polynucleotides are selected from the group consisting of: polynucleotides comprising SNPs; polynucleotides comprising alleles of SNPs; protein coding DNA or RNA polynucleotides; non-protein coding DNA or RNA polynucleotides; polynucleotides comprising mutations; polynucleotides comprising deletions; and polynucleotides comprising insertions.

In some embodiments, a plurality of target polynucleotides are detected in a single reaction. In some embodiments, the target polynucleotides comprise at least two polynucleotides selected from the group consisting of: polynucleotides comprising SNPs; polynucleotides comprising alleles of SNPs; protein coding DNA or RNA polynucleotides; non-protein coding DNA or RNA polynucleotides; polynucleotides comprising mutations; polynucleotides comprising deletions; and polynucleotides comprising insertions.

In some embodiments, the target polynucleotides comprise at least one polynucleotide selected from the group consisting of: polynucleotides comprising SNPs and polynucleotides comprising alleles of SNPs; and at least one polynucleotide selected from the group consisting of: protein coding DNA or RNA polynucleotides; non-protein coding DNA or RNA polynucleotides; polynucleotides comprising mutations; polynucleotides comprising deletions; and polynucleotides comprising insertions.

In some embodiments, at least one of the target polynucleotides comprises a SNP and wherein the method genotypes the nucleotide at the SNP. In some embodiments, a dual domain or truncated dual domain primers specific for each of 2 or more alleles of a target SNP are present in the same reaction. In some embodiments, one dual domain or truncated dual domain primer of a set is specific for the predominant allele of the target SNP.

In some embodiments, the dual domain or truncated dual domain primers specific for each of 2 or more alleles or sequence variants of a target nucleotide are present in the same reaction. In some embodiments, one dual domain or truncated dual domain primer of a set is specific for the predominant allele or variant allele of the target polynucleotide.

In some embodiments, the amplified products of two or more members of a primer subset can be distinguished. In some embodiments, the amplified products of two or more members of a primer subset are distinguished by being of distinct sizes. In some embodiments, the amplified products of two or more members of a primer subset are distinguished by being labeled with different detectable labels. In some embodiments, the products of two or more members of a primer subset are distinguished by sequencing the products. In some embodiments, the products of two or more members of a primer subset are distinguished by oligonucleotide hybridization. In some embodiments, the products of two or more members of a primer subset are distinguished by melting curve analysis of the amplified products.

In some embodiments, the core sequence is 10 to 40 nucleotides in length. In some embodiments, the core sequence is 11 to 20 nucleotides in length.

In some embodiments, the first phase comprises from 1 to 10 cycles. In some embodiments, the first phase comprises from 3 to 5 cycles.

In some embodiments, each 5′ tail sequence of a set has fewer than 3 contiguous homologous bases relative to the other 5′ tail sequences of the set. In some embodiments, each 5′ tail sequence of a set has fewer than 2 contiguous homologous bases relative to the other 5′ tail sequences of the set. In some embodiments, the 5′ tail sequence is from 1 to 150 nucleotides in length. In some embodiments, the 5′ tail sequence is from 1 to 50 nucleotides in length.

In some embodiments, the second annealing temperature is at least 4° C. higher than the first annealing temperature. In some embodiments, the dual domain or truncated dual domain primers present in a reaction have Tₘ values for their respective 3′ core regions that vary by no more than 8° C. from each other.

In one aspect, the invention described herein relates to a composition for determining the presence of one or more target polynucleotides, comprising; at least one set of oligonucleotide primers specific for each target polynucleotide; wherein each set of oligonucleotide primers comprises a first subset of at least one truncated dual domain forward primer and a second subset of at least one reverse primer; wherein each truncated dual domain primer of a set comprises a 5′ tail region that differs from the 5′ tail region on other truncated dual domain primers in the set and a 3′ core region complementary to a sequence on one strand of a double-stranded nucleic acid comprising the target; wherein for each truncated dual domain primer, the 3′ core substantially anneals to its complementary target site sequence at a first annealing temperature, and the sequence comprised by the 5′ tail and 3′core region substantially anneals to its complement at a second annealing temperature, the second annealing temperature being higher than the first annealing temperature, such that at the second annealing temperature the 3′ core of the truncated dual domain primer cannot substantially anneal to a template molecule that does not also have the complement of the primer's 5′ tail sequence; wherein for each member of the truncated dual domain primer set: the 5′ tail sequence does not have any homology to the target sequence; and the 5′ tail sequence has 6 or fewer contiguous homologous bases relative to the other 5′ tail sequences of the truncated dual domain primer set.

In some embodiments, the composition can further comprise a nucleic acid sample.

In some embodiments, at least one forward primer of the composition comprises a dual domain primer; wherein each dual domain primer of a set comprises a 5′ tail region that differs from the 5′ tail region on other dual domain primers in the set, a 3′ core region complementary to a sequence on one strand of a double-stranded nucleic acid comprising said target, and a terminal nucleotide complementary to one of the variant nucleotides occurring at said target site; wherein for each dual domain primer, the 3′ core substantially anneals to its complementary target site sequence at a first annealing temperature, and the sequence comprised by the 5′ tail and 3′ core region substantially anneals to its complement at a second annealing temperature, the second annealing temperature being higher than the first annealing temperature, such that at said second annealing temperature said 3′ core of said dual domain primer cannot substantially anneal to a template molecule that does not also have the complement of the primer's 5′ tail sequence; wherein for each member of said dual domain primer set: the 5′ tail sequence does not have any homology to the target sequence; and the 5′ tail sequence has 6 or fewer contiguous homologous bases relative to the other 5′ tail sequences of the dual domain primer set.

In some embodiments, a reverse primer comprises a dual domain primer. In some embodiments, a reverse primer comprises a truncated dual domain primer. In some embodiments, a reverse primer comprises an amplifying primer.

In some embodiments, the target polynucleotides are selected from the group consisting of polynucleotides comprising SNPs; polynucleotides comprising alleles of SNPs; protein coding DNA or RNA polynucleotides; non-protein coding DNA or RNA polynucleotides; polynucleotides comprising mutations; polynucleotides comprising deletions; and polynucleotides comprising insertions. In some embodiments, the target polynucleotides comprise at least two polynucleotides selected from the group consisting of: polynucleotides comprising SNPs; polynucleotides comprising alleles of SNPs; protein coding DNA or RNA polynucleotides; non-protein coding DNA or RNA polynucleotides; polynucleotides comprising mutations; polynucleotides comprising deletions; and polynucleotides comprising insertions. In some embodiments, the target polynucleotides comprise at least one polynucleotide selected from the group consisting of: polynucleotides comprising SNPs and polynucleotides comprising alleles of SNPs; and at least one polynucleotide selected from the group consisting of: protein coding DNA or RNA polynucleotides; non-protein coding DNA or RNA polynucleotides; polynucleotides comprising mutations; polynucleotides comprising deletions; and polynucleotides comprising insertions.

In some embodiments, a subset of dual domain forward primers comprises primers specific for each of 2 or more alleles of a target SNP. In some embodiments, one dual domain forward primer of a set is specific for the predominant allele of the target SNP.

In some embodiments, the subset of dual domain or truncated dual domain forward primers comprises primers specific for each of 2 or more alleles or sequence variants of a target nucleotide. In some embodiments, one dual domain or truncated dual domain forward primer of a set is specific for the predominant allele or variant allele of the target polynucleotide.

In some embodiments, the core sequence is 10 to 40 nucleotides in length. In some embodiments, the core sequence is 11 to 20 nucleotides in length.

In some embodiments, each 5′ tail sequence of a set has fewer than 3 contiguous homologous bases relative to the other 5′ tail sequences of the set. In some embodiments, each 5′ tail sequence of a set has fewer than 2 contiguous homologous bases relative to the other 5′ tail sequences of the set. In some embodiments, the 5′ tail sequence is from 1 to 150 nucleotides in length. In some embodiments, the 5′ tail sequence is from 1 to 50 nucleotides in length.

In some embodiments, the second annealing temperature is at least 4° C. higher than the first annealing temperature. In some embodiments, the dual domain primers present in a reaction have Tₘ values for their respective 3′ core regions that vary by no more than 8° C. from each other.

One aspect of the invention described herein relates to a method for determining the presence of one or more target polynucleotides, the method comprising; performing a PCR amplification regimen comprising cycles of strand separation, primer annealing, and primer extension on a reaction mixture comprising a nucleic acid sample and a set of oligonucleotide primers specific for each target polynucleotide; wherein each set of oligonucleotide primers comprises a first subset of at least one truncated dual domain forward primer and a second subset of at least one reverse primer; wherein each truncated dual domain primer of a set comprises a 5′ tail region that differs from the 5′ tail region on other truncated dual domain primers in the set and a 3′ core region complementary to a sequence on one strand of a double-stranded nucleic acid comprising the target; wherein for each truncated dual domain primer, the 3′ core substantially anneals to its complementary target site sequence at a first annealing temperature, and the sequence comprised by the 5′ tail and 3′ core region substantially anneals to its complement at a second annealing temperature, the second annealing temperature being higher than the first annealing temperature, such that at the second annealing temperature the 3′ core of the truncated dual domain primer cannot substantially anneal to a template molecule that does not also have the complement of the truncated dual domain primer's 5′ tail sequence; wherein for each truncated dual domain primer of a primer set: the 5′ tail sequence does not have any homology to the target sequence; and the 5′ tail sequence has 6 or fewer contiguous homologous bases relative to any other 5′ tail sequence of the truncated dual domain primer set; wherein the PCR amplification regimen comprises first and second phases, the first phase comprising annealing at the first annealing temperature for a first set of cycles, and; the second phase comprising annealing at the second annealing temperature for a second set of cycles; and detecting an amplified product for each target polynucleotide, wherein the detecting indicates the presence of the target polynucleotide.

In some embodiments, a reverse primer comprises a dual domain primer. In some embodiments, a reverse primer comprises a truncated dual domain primer. In some embodiments, a reverse primer comprises an amplifying primer.

In some embodiments, the set of primers comprises at least one dual domain forward primer; wherein each dual domain primer of a set comprises a 5′ tail region that differs from the 5′ tail region of other dual domain primers in the set, a 3′ core region complementary to a sequence on one strand of a double-stranded nucleic acid comprising said target, and a terminal nucleotide complementary to one of the variant nucleotides occurring at said target site; wherein for each dual domain primer, the 3′ core substantially anneals to its complementary target site sequence at a first annealing temperature, and the sequence comprised by the 5′ tail and 3′ core region substantially anneals to its complement at a second annealing temperature, the second annealing temperature being higher than the first annealing temperature, such that at said second annealing temperature said 3′ core of said dual domain primer cannot substantially anneal to a template molecule that does not also have the complement of the dual domain primer's 5′ tail sequence; and wherein for each truncated dual domain primer of a primer set: the 5′ tail sequence does not have any homology to the target sequence; and the 5′ tail sequence has 6 or fewer contiguous homologous bases relative to any other 5′ tail sequence of the dual domain or truncated dual domain primer set.

In some embodiments, the target polynucleotides are selected from the group consisting of: polynucleotides comprising SNPs; polynucleotides comprising alleles of SNPs; protein coding DNA or RNA polynucleotides; non-protein coding DNA or RNA polynucleotides; polynucleotides comprising mutations; polynucleotides comprising deletions; and polynucleotides comprising insertions.

In some embodiments, a plurality of target polynucleotides are detected in a single reaction. In some embodiments, the target polynucleotides comprise at least two polynucleotides selected from the group consisting of: polynucleotides comprising SNPs; polynucleotides comprising alleles of SNPs; protein coding DNA or RNA polynucleotides; non-protein coding DNA or RNA polynucleotides; polynucleotides comprising mutations; polynucleotides comprising deletions; and polynucleotides comprising insertions.

In some embodiments, the target polynucleotides comprise at least one polynucleotide selected from the group consisting of: polynucleotides comprising SNPs and polynucleotides comprising alleles of SNPs; and at least one polynucleotide selected from the group consisting of: protein coding DNA or RNA polynucleotides; non-protein coding DNA or RNA polynucleotides; polynucleotides comprising mutations; polynucleotides comprising deletions; and polynucleotides comprising insertions.

In some embodiments, at least one of the target polynucleotides comprises a SNP and wherein the method genotypes the nucleotide at the SNP. In some embodiments, a dual domain or truncated dual domain primers specific for each of 2 or more alleles of a target SNP are present in the same reaction. In some embodiments, one dual domain or truncated dual domain primer of a set is specific for the predominant allele of the target SNP.

In some embodiments, the dual domain or truncated dual domain primers specific for each of 2 or more alleles or sequence variants of a target nucleotide are present in the same reaction. In some embodiments, one dual domain or truncated dual domain primer of a set is specific for the predominant allele or variant allele of the target polynucleotide.

In some embodiments, the amplified products of two or more members of a primer subset can be distinguished. In some embodiments, the amplified products of two or more members of a primer subset are distinguished by being of distinct sizes. In some embodiments, the amplified products of two or more members of a primer subset are distinguished by being labeled with different detectable labels. In some embodiments, the products of two or more members of a primer subset are distinguished by sequencing the products. In some embodiments, the products of two or more members of a primer subset are distinguished by oligonucleotide hybridization. In some embodiments, the products of two or more members of a primer subset are distinguished by melting curve analysis of the amplified products.

In some embodiments, the core sequence is 10 to 40 nucleotides in length. In some embodiments, the core sequence is 11 to 20 nucleotides in length.

In some embodiments, the first phase comprises from 1 to 10 cycles. In some embodiments, the first phase comprises from 3 to 5 cycles.

In some embodiments, each 5′ tail sequence of a set has fewer than 3 contiguous homologous bases relative to the other 5′ tail sequences of the set. In some embodiments, each 5′ tail sequence of a set has fewer than 2 contiguous homologous bases relative to the other 5′ tail sequences of the set. In some embodiments, the 5′ tail sequence is from 1 to 150 nucleotides in length. In some embodiments, the 5′ tail sequence is from 1 to 50 nucleotides in length.

In some embodiments, the second annealing temperature is at least 4° C. higher than the first annealing temperature. In some embodiments, the dual domain or truncated dual domain primers present in a reaction have Tₘ values for their respective 3′ core regions that vary by no more than 8° C. from each other.

In one aspect, the invention described herein relates to a composition for determining the presence of one or more target polynucleotides, comprising; at least one set of oligonucleotide primers specific for each target polynucleotide; wherein each set of oligonucleotide primers comprises a first subset of at least one truncated dual domain forward primer and a second subset of at least one reverse primer; wherein each truncated dual domain primer of a set comprises a 5′ tail region that differs from the 5′ tail region on other truncated dual domain primers in the set and a 3′ core region complementary to a sequence on one strand of a double-stranded nucleic acid comprising the target; wherein for each truncated dual domain primer, the 3′ core substantially anneals to its complementary target site sequence at a first annealing temperature, and the sequence comprised by the 5′ tail and 3′ core region substantially anneals to its complement at a second annealing temperature, the second annealing temperature being higher than the first annealing temperature, such that at the second annealing temperature the 3′ core of the truncated dual domain primer cannot substantially anneal to a template molecule that does not also have the complement of the primer's 5′ tail sequence; wherein for each member of the truncated dual domain primer set: the 5′ tail sequence does not have any homology to the target sequence; and the 5′ tail sequence has 6 or fewer contiguous homologous bases relative to the other 5′ tail sequences of the truncated dual domain primer set.

In some embodiments, the composition can further comprise a nucleic acid sample.

In some embodiments, at least one forward primer of the composition comprises a dual domain primer; wherein each dual domain primer of a set comprises a 5′ tail region that differs from the 5′ tail region on other dual domain primers in the set, a 3′ core region complementary to a sequence on one strand of a double-stranded nucleic acid comprising said target, and a terminal nucleotide complementary to one of the variant nucleotides occurring at said target site; wherein for each dual domain primer, the 3′ core substantially anneals to its complementary target site sequence at a first annealing temperature, and the sequence comprised by the 5′ tail and 3′ core region substantially anneals to its complement at a second annealing temperature, the second annealing temperature being higher than the first annealing temperature, such that at said second annealing temperature said 3′ core of said dual domain primer cannot substantially anneal to a template molecule that does not also have the complement of the primer's 5′ tail sequence; wherein for each member of said dual domain primer set: the 5′ tail sequence does not have any homology to the target sequence; and the 5′ tail sequence has 6 or fewer contiguous homologous bases relative to the other 5′ tail sequences of the dual domain primer set.

In some embodiments, a reverse primer comprises a dual domain primer. In some embodiments, a reverse primer comprises a truncated dual domain primer. In some embodiments, a reverse primer comprises an amplifying primer.

In some embodiments, the target polynucleotides are selected from the group consisting of polynucleotides comprising SNPs; polynucleotides comprising alleles of SNPs; protein coding DNA or RNA polynucleotides; non-protein coding DNA or RNA polynucleotides; polynucleotides comprising mutations; polynucleotides comprising deletions; and polynucleotides comprising insertions. In some embodiments, the target polynucleotides comprise at least two polynucleotides selected from the group consisting of: polynucleotides comprising SNPs; polynucleotides comprising alleles of SNPs; protein coding DNA or RNA polynucleotides; non-protein coding DNA or RNA polynucleotides; polynucleotides comprising mutations; polynucleotides comprising deletions; and polynucleotides comprising insertions. In some embodiments, the target polynucleotides comprise at least one polynucleotide selected from the group consisting of: polynucleotides comprising SNPs and polynucleotides comprising alleles of SNPs; and at least one polynucleotide selected from the group consisting of: protein coding DNA or RNA polynucleotides; non-protein coding DNA or RNA polynucleotides; polynucleotides comprising mutations; polynucleotides comprising deletions; and polynucleotides comprising insertions.

In some embodiments, a subset of dual domain forward primers comprises primers specific for each of 2 or more alleles of a target SNP. In some embodiments, one dual domain forward primer of a set is specific for the predominant allele of the target SNP.

In some embodiments, the subset of dual domain or truncated dual domain forward primers comprises primers specific for each of 2 or more alleles or sequence variants of a target nucleotide. In some embodiments, one dual domain or truncated dual domain forward primer of a set is specific for the predominant allele or variant allele of the target polynucleotide.

In some embodiments, the core sequence is 10 to 40 nucleotides in length. In some embodiments, the core sequence is 11 to 20 nucleotides in length.

In some embodiments, each 5′ tail sequence of a set has fewer than 3 contiguous homologous bases relative to the other 5′ tail sequences of the set. In some embodiments, each 5′ tail sequence of a set has fewer than 2 contiguous homologous bases relative to the other 5′ tail sequences of the set. In some embodiments, the 5′ tail sequence is from 1 to 150 nucleotides in length. In some embodiments, the 5′ tail sequence is from 1 to 50 nucleotides in length.

In some embodiments, the second annealing temperature is at least 4° C. higher than the first annealing temperature. In some embodiments, the dual domain primers present in a reaction have Tₘ values for their respective 3′ core regions that vary by no more than 8° C. from each other.

### BRIEF DESCRIPTION OF THE DRAWINGS

FIG. 1 is a schematic depiction of a PCR amplification regimen using traditional PCR primers.

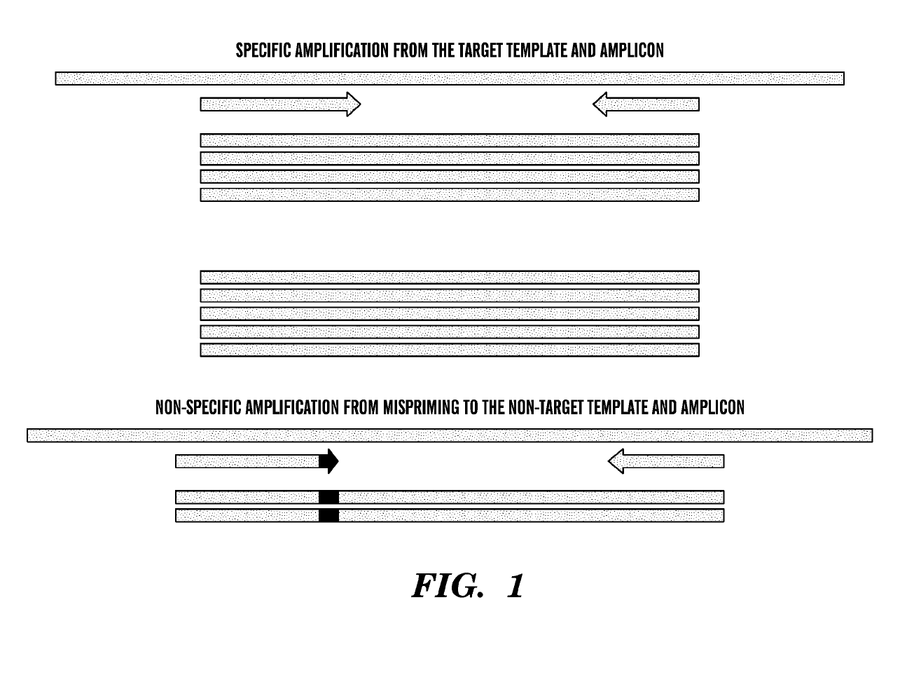

FIG. 2 is a schematic depiction of a PCR amplification regimen using short primers with identical or highly similar tail sequences.

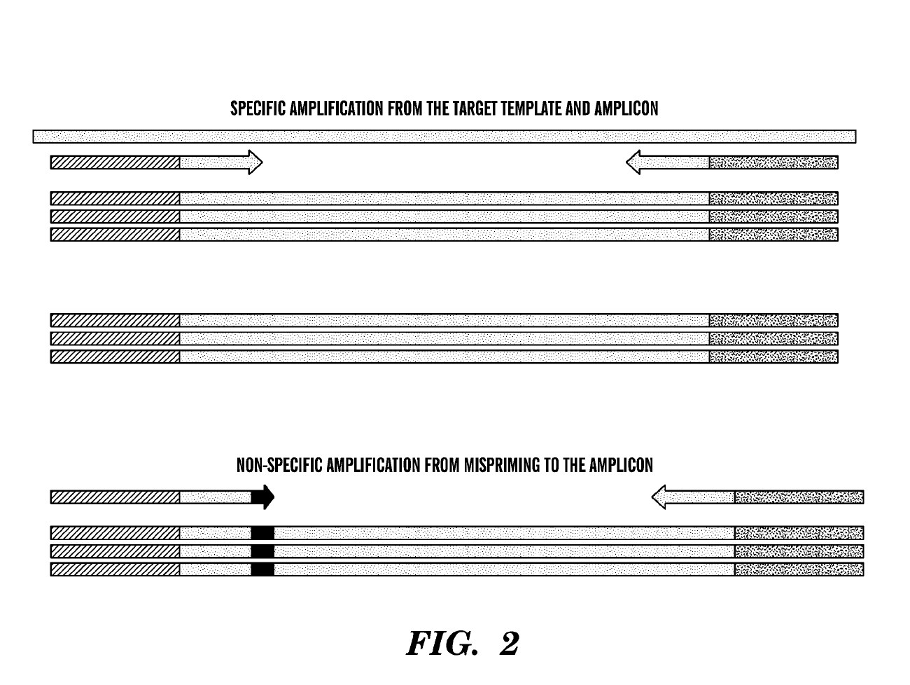

FIG. 3 is a schematic depiction of a PCR amplification regimen using short primers with highly heterogeneous tail sequences.

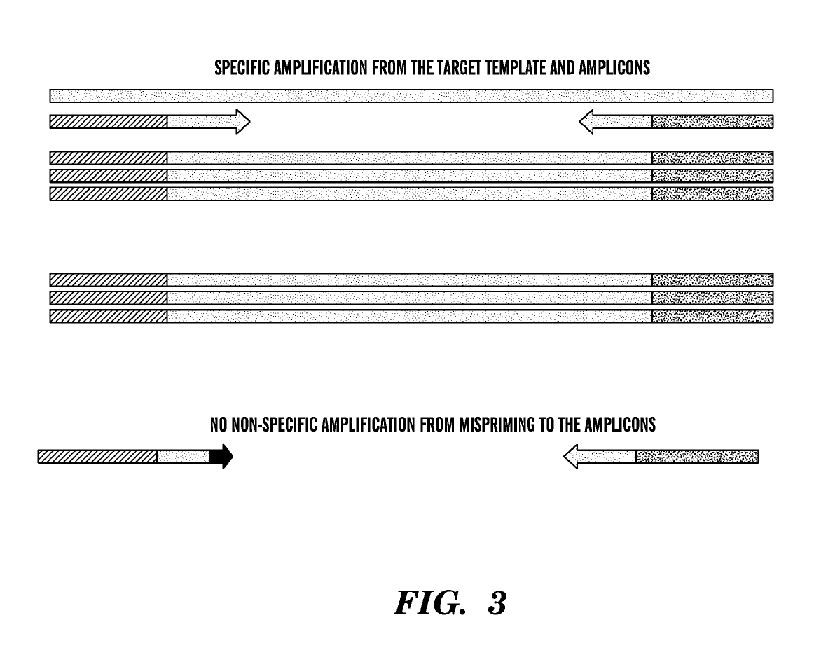

FIG. 4 depicts the results of a multiplex KRAS/BRAF Mutation Assay with individual mutant (1 ng) and K562 wildtype (50 ng) cell line DNA.

FIG. 5 depicts the results of a multiplex KRAS/BRAF Mutation Assay with individual mutant cell line DNA (1%) mixed with K562 wildtype cell line DNA.

FIG. 6 depicts the results of a multiplex IL-10 (−1082)/TNF-α (−308) SNP Assay with synthetic DNA and purified human DNA samples.

FIG. 7 depicts a schematic of an example of a set of oligonucleotide primers and their relation to two alleles of a target locus. The set of primers comprises two subsets. The first subset comprises a single amplifying primer (dashed black line) capable of annealing to both alleles of the target locus and amplifying the targets in combination with either of the primers of the second subset. The second subset comprises two dual domain primers. The 3′ core is shown as a solid black block and the 5′ tail as a block with vertical lines. The 3′ nucleotide of the first dual domain primer is complementary to allele #1 of the target locus, while the 3′ nucleotide of the second dual domain primer is complementary to allele #2 of the target locus.

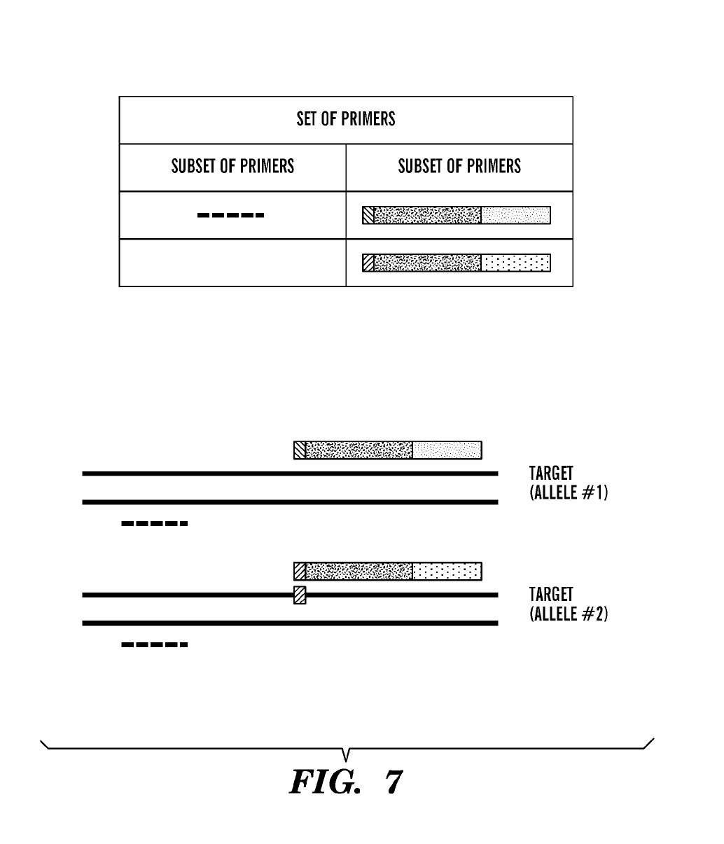

FIG. 8 depicts the results of a KRAS SNP spike-in plasma sample assay.

FIG. 9 depicts a schematic of exemplary embodiments of primers for use in detecting a SNP in codons 12 and 13 of KRAS. The target sequence (SEQ ID NO: 85) is shown in italics. A 3′ core sequence for use in a dual domain primer is shown in black from 3′ to 5′ (SEQ ID NO: 86) and a plurality of possible reverse primers are shown 5′ to 3′ (SEQ ID NOs: 87-98).

FIG. 10 depicts a schematic of exemplary embodiments of primers for use in detecting a SNP in codon 61 of KRAS. The target sequence (SEQ ID NO: 99) is shown in italics. A 3′ core sequence for use in a dual domain primer is shown in black from 3′ to 5′ (SEQ ID NO: 100) and a plurality of possible reverse primers are shown 5′ to 3′ (SEQ ID NOs: 101-109).

FIG. 11 depicts a schematic of exemplary embodiments of primers for use in detecting a SNP in codon 146 of KRAS. The target sequence (SEQ ID NO: 110) is shown in italics. A 3′ core sequence for use in a dual domain primer is shown in black from 3′ to 5′ (SEQ ID NO: 111) and a plurality of possible reverse primers are shown 5′ to 3′ (SEQ ID NOs: 112-119).

FIG. 12 depicts a schematic of exemplary embodiments of primers for use in detecting BRAF V600. The target sequence (SEQ ID NO: 120) is shown in italics. A 3′ core sequence for use in a dual domain primer is shown in black from 3′ to 5′ (SEQ ID NO: 121) and a plurality of possible reverse primers are shown 5′ to 3′ (SEQ ID NOs: 122-129).

FIG. 13 depicts a schematic of exemplary embodiments of primers for use in detecting a SNP in codons 12 and 13 of KRAS on the complementary strand of that shown in FIG. 9. The target sequence (SEQ ID NO: 130) is shown in italics. A 3′ core sequence for use in a dual domain primer is shown in black from 5′ to 3′ (SEQ ID NO: 131) and a plurality of possible reverse primers are shown 3′ to 5′(SEQ ID NOs: 132-138).

FIG. 14 depicts a schematic of exemplary embodiments of primers for use in detecting a SNP in codon 61 of KRAS on the complementary strand of that shown in FIG. 10. The target sequence (SEQ ID NO: 139) is shown in italics. A 3′ core sequence for use in a dual domain primer is shown in black from 5′ to 3′ (SEQ ID NO: 140) and a plurality of possible reverse primers are shown 3′ to 5 (SEQ ID NOs: 141-146).

FIG. 15 depicts a schematic of exemplary embodiments of primers for use in detecting a SNP in codon 146 of KRAS on the complementary strand of that shown in FIG. 11. The target sequence (SEQ ID NO: 147) is shown in italics. A 3′ core sequence for use in a dual domain primer is shown in black from 5′ to 3′ (SEQ ID NO: 148) and a plurality of possible reverse primers are shown 3′ to 5′ (SEQ ID NOs: 149-154).

FIG. 16 depicts a schematic of exemplary embodiments of primers for use in detecting BRAF V600 on the complementary strand of that shown in FIG. 12. The target sequence (SEQ ID NO: 155) is shown in italics. A 3′ core sequence for use in a dual domain primer is shown in black from 5′ to 3′ (SEQ ID NO: 156) and a plurality of possible reverse primers are shown 3′ to 5′ (SEQ ID NOs: 157-162).

### DETAILED DESCRIPTION OF THE INVENTION

Described herein are methods and compositions related to determining the presence of one or more target polynucleotides in a nucleic acid sample. The methods and compositions described herein are based at least in part upon the inventor's discovery of an improved method for performing multiplex, multimodal and/or genotyping PCR amplification regimens via the use of dual domain primers.

### DEFINITIONS

For convenience, certain terms employed herein, in the specification, examples and appended claims are collected here. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

Definitions of common terms in cell biology and molecular biology can be found in The Encyclopedia of Molecular Biology, published by Blackwell Science Ltd., 1994 (ISBN 0-632-02182-9). Definitions of common terms in molecular biology can also be found in Benjamin Lewin, Genes X, published by Jones & Bartlett Publishing, 2009 (ISBN-10: 0763766321); Kendrew et al. (eds.) and Molecular Biology and Biotechnology: a Comprehensive Desk Reference, published by VCH Publishers, Inc., 1995 (ISBN 1-56081-569-8).

Unless otherwise stated, the present invention was performed using standard procedures, as described, for example in Sambrook et al., Molecular Cloning: A Laboratory Manual (3 ed.), Cold Spring Harbor Laboratory Press, Cold Spring Harbor, N.Y., USA (2001); Davis et al., Basic Methods in Molecular Biology, Elsevier Science Publishing, Inc., New York, USA (1995) which are all incorporated by reference herein in their entireties.

The term “isolated” or “partially purified” as used herein refers, in the case of a nucleic acid, to a nucleic acid separated from at least one other component (e.g., nucleic acid or polypeptide) that is present with the nucleic acid as found in its natural source and/or that would be present with the nucleic acid when expressed by a cell. A chemically synthesized nucleic acid or one synthesized using in vitro transcription/translation is considered “isolated.”

As used herein, the term “nucleic acid” or “nucleic acid sequence” refers to a polymeric molecule incorporating units of ribonucleic acid, deoxyribonucleic acid or an analog thereof. The nucleic acid can be either single-stranded or double-stranded. A single-stranded nucleic acid can be one strand of a denatured double-stranded DNA. Alternatively, it can be a single-stranded nucleic acid not derived from any double-stranded DNA. In one aspect, a template nucleic acid is DNA. In another aspect, a template is RNA. Suitable nucleic acid molecules include DNA, including genomic DNA and cDNA. Other suitable nucleic acid molecules include RNA, including mRNA, rRNA and tRNA. The nucleic acid molecule can be naturally occurring, as in genomic DNA, or it may be synthetic, i.e., prepared based upon human action, or may be a combination of the two. The nucleic acid molecule can also have certain modifications such as 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA), cholesterol addition, and phosphorothioate backbone as described in US Patent Application 20070213292; and certain ribonucleosides that are linked between the 2′-oxygen and the 4′-carbon atoms with a methylene unit as described in U.S. Pat. No. 6,268,490, wherein both patent and patent application are incorporated herein by reference in their entirety.

The term “gene” means a nucleic acid sequence which is transcribed (DNA) to RNA in vitro or in vivo when operably linked to appropriate regulatory sequences. The gene can include regulatory regions preceding and following the coding region, e.g. 5′ untranslated (5′UTR) or “leader” sequences and 3′ UTR or “trailer” sequences, as well as intervening sequences (introns) between individual coding segments (exons).

As used herein, the term “complementary” refers to the hierarchy of hydrogen-bonded base pair formation preferences between the nucleotide bases G, A, T, C and U, such that when two given polynucleotides or polynucleotide sequences anneal to each other, A pairs with T and G pairs with C in DNA, and G pairs with C and A pairs with U in RNA. As used herein, “substantially complementary” refers to a primer having at least 90% complementarity over the entire length of a primer with a second nucleotide sequence, e.g. 90% complementary, 95% complementary, 98% complementary, 99% complementary, or 100% complementary.

As used herein the term “comprising” or “comprises” is used in reference to compositions, methods, and respective component(s) thereof, that are essential to the method or composition, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Other than in the operating examples, or where otherwise indicated, all numbers expressing quantities of ingredients or reaction conditions used herein should be understood as modified in all instances by the term “about.” The term “about” when used in connection with percentages can mean±1%.

Other terms are defined herein within the description of the various aspects of the invention.

In some embodiments, the methods and compositions described herein relate to PCR amplification regimens having reduced background and increased specificity compared to commonly applied methods. One method of increasing the specificity and decreasing background levels in a PCR amplification of a target sequence involves shortening the length of a primer. As a primer decreases in length, the effect of mispairing at any one position upon the melting temperature of the nucleic acid primer-template duplex increases (see Table 1). However, even with short primers, the problem of amplification of non-target sequences (i.e. background noise) is still non-trivial. Coupling a 5′ tail region to the short target-specific primer sequences to generate a dual domain primer according to the rules described herein ensures that during the second phase of the PCR amplification regimen, the primers are not amplifying sequences from the nucleic acid sample, but only from the amplification products generated during the first phase of the PCR amplification regimen. This avoids one ongoing source of background amplification. By keeping the number of cycles in the first phase of the PCR amplification regimen to a minimum, the amount of background amplification is significantly reduced. In preferred embodiments, the amount of background amplification is reduced below detectable levels. In order to avoid cross-talk between primers in the second phase of the PCR amplification regimen, it is preferred that the 5′ tail region of each dual domain primer and/or truncated dual domain primer in a primer set differs from the 5′ tail regions of every other dual domain primer and/or truncated dual domain primer in the set.

Described herein are methods and compositions for detecting and/or analyzing one or more target polynucleotides in a nucleic acid sample. In some embodiments, the methods described herein relate to the use of at least one dual domain primer (or truncated dual domain primer) in a PCR amplification regimen having at least two phases which are differentiated by having different annealing temperatures. During the first phase of the PCR amplification regimen, a 3′ core region of the dual domain primer (or truncated dual domain primer) specifically anneals to its target and primer extension occurs, creating amplification products comprising the target polynucleotide sequence and/or its complement. The amplification product will contain both the target sequence which is being amplified and a “tag” at at least one end of the product, the tag comprising the sequence of the 5′ tail of the dual domain (or truncated dual domain primer). In the second phase of the PCR amplification regimen, the annealing temperature is raised, such that sequence complementary to both the 5′ tail region and 3′ core region is required for annealing of the dual domain primer (or truncated dual domain primer) to a target, thus preventing the primer from mispriming to off-target polynucleotides.

Considerations for performing the subject methods are described herein below. In short, it is important to consider aspects of PCR generally, as well as aspects more specifically applicable to the claimed method, for example, dual domain primer design, annealing temperatures, cycling parameters for each phase of amplification, target-specific considerations including considerations for SNP detection, multimodal amplification, options for multiplexing, and various considerations for detection of amplified products.

PCR

The methods and compositions described herein relate to performing a polymerase chain reaction (PCR) amplification regimen. As used herein, the term “amplification regimen” refers to a process of specifically amplifying, i.e., increasing the abundance of, a nucleic acid sequence of interest, and more particularly, the exponential amplification occurring when the products of a previous polymerase extension serve as templates for the successive rounds of extension. A PCR amplification regimen according to the invention comprises at least two, and preferably at least 5, 10, 15, 20, 25, 30, 35 or more iterative cycles, where each cycle comprises the steps of: 1) strand separation (e.g., thermal denaturation); 2) oligonucleotide primer annealing to template molecules; and 3) nucleic acid polymerase extension of the annealed primers. Conditions and times necessary for each of these steps can be devised by one of ordinary skill in the art. An amplification regimen according to the methods described herein is preferably performed in a thermal cycler, many of which are commercially available.

PCR requires the use of a nucleic acid polymerase. As used herein, the phrase “nucleic acid polymerase” refers an enzyme that catalyzes the template-dependent polymerization of nucleoside triphosphates to form primer extension products that are complementary to the template nucleic acid sequence. A nucleic acid polymerase enzyme initiates synthesis at the 3′ end of an annealed primer and proceeds in the direction toward the 5′ end of the template. Numerous nucleic acid polymerases are known in the art and commercially available. One group of preferred nucleic acid polymerases are thermostable, i.e. they retain function after being subjected to temperatures sufficient to denature annealed strands of complementary nucleic acids. e.g. 94° C., or sometimes higher.

As understood in the art, PCR requires cycles including a strand separation step generally involving heating of the reaction mixture. As used herein, the term “strand separation” or “separating the strands” means treatment of a nucleic acid sample such that complementary double-stranded molecules are separated into two single strands available for annealing to an oligonucleotide primer. More specifically, strand separation according to the methods described herein is achieved by heating the nucleic acid sample above its Tₘ. Generally, for a sample containing nucleic acid molecules in buffer suitable for a nucleic acid polymerase, heating to 94° C. is sufficient to achieve strand separation. An exemplary buffer contains 50 mM KCl, 10 mM Tric-HCl (pH 8.8@25° C.), 0.5 to 3 mM MgCl2, and 0.1% BSA.

As also understood in the art, PCR requires annealing primers to template nucleic acids. As used herein, “anneal” refers to permitting two complementary or substantially complementary nucleic acids strands to hybridize, and more particularly, when used in the context of PCR, to hybridize such that a primer extension substrate for a template-dependent polymerase enzyme is formed. Conditions for primer-target nucleic acid annealing vary with the length and sequence of the primer and are based upon the calculated Tₘ for the primer. Generally, an annealing step in an amplification regimen involves reducing the temperature following the strand separation step to a temperature based on the calculated Tₘ for the primer sequence, for a time sufficient to permit such annealing.

Tₘ can be readily predicted by one of skill in the art using any of a number of widely available algorithms (e.g. OLIGO™ (Molecular Biology Insights Inc. Colorado) primer design software and VENTRO NTI™ (Invitrogen, Inc. California) primer design software and programs available on the internet, including Primer3 and Oligo Calculator). For the methods described in the Examples provided herein, the Tₘ's were calculated using the NetPrimer software (Premier Biosoft; Palo Alto, Calif.; and freely available on the world wide web at http://www.premierbiosoft.com/netprimer/netprlaunch/Help/xnetprlaunch.html). The Tₘ of a primer can be calculated using following formula, which is used by NetPrimer software and is described in more detail in Frieir et al. PNAS 1986 83:9373-9377 which is incorporated by reference herein in its entirety.

Tₘ=ΔH/(ΔS+R*ln(C/4))+16.6 log([K⁺]/(1+0.7[K*]))−273.15

wherein, ΔH is enthalpy for helix formation; ΔS is entropy for helix formation; R is molar gas constant (1.987 cal/° C.*mol); C is the nucleic acid concentration; and [K*] is salt concentration. For most amplification regimens, the annealing temperature is selected to be about 5° C. below the predicted Tₘ, although temperatures closer to and above the Tₘ (e.g. between 1° C. and 5° C. below the predicted Tₘ or between 1° C. and 5° C. above the predicted Tₘ) can be used, as can, for example, temperatures more than 5° C. below the predicted Tₘ (e.g. 6° C. below, 8° C. below, 10° C. below or lower). Generally, the closer the annealing temperature is to the Tₘ, the more specific is the annealing. The time allowed for primer annealing during a PCR amplification regimen depends largely upon the volume of the reaction, with larger volumes requiring longer times, but also depends upon primer and template concentrations, with higher relative concentrations of primer to template requiring less time than lower relative concentrations. Depending upon volume and relative primer/template concentration, primer annealing steps in an amplification regimen can be on the order of 1 second to 5 minutes, but will generally be between 10 seconds and 2 minutes, preferably on the order of 30 seconds to 2 minutes.

As used herein, “substantially anneal” refers to a degree of annealing during a PCR amplification regimen which is sufficient to produce a detectable level of a specifically amplified product.

PCR also relies upon polymerase extension of annealed primers at each cycle. As used herein, the term “polymerase extension” means the template-dependent incorporation of at least one complementary nucleotide, by a nucleic acid polytmerase, onto the 3′ end of an annealed primer. Polymerase extension preferably adds more than one nucleotide, preferably up to and including nucleotides corresponding to the full length of the template. Conditions for polymerase extension vary with the identity of the polymerase. The temperature used for polymerase extension is generally based upon the known activity properties of the enzyme. Although, where annealing temperatures are required to be, for example, below the optimal temperatures for the enzyme, it will often be acceptable to use a lower extension temperature. In general, although the enzymes retain at least partial activity below their optimal extension temperatures, polymerase extension by the most commonly used thermostable polymerases (e.g., Taq polymerase and variants thereof) is performed at 65° C. to 75° C., preferably about 68-72° C.

Primer extension is performed under conditions that permit the extension of annealed oligonucleotide primers. As used herein, the term “conditions that permit the extension of an annealed oligonucleotide such that extension products are generated” refers to the set of conditions including, for example temperature, salt and co-factor concentrations, pH, and enzyme concentration under which a nucleic acid polymerase catalyzes primer extension. Such conditions will vary with the identity of the nucleic acid polymerase being used, but the conditions for a large number of useful polymerase enzymes are well known to those skilled in the art. One exemplary set of conditions is 50 mM KCl, 10 mM Tric.HCl (pH 8.8@25° C.), 0.5 to 3 mM MgCl2, 200 uM each dNTP, and 0.1% BSA at 72° C. under which Taq polymerase catalyzes primer extension.

As used herein, “amplified product” refers to polynucleotides resulting from a PCR reaction that are copies of a portion of a particular target polynucleotide sequence and/or its complementary sequence, which correspond in nucleotide sequence to the template polynucleotide sequence and/or its complementary sequence. An amplification product can further comprise sequence specific to the primers and which flanks sequence which is a portion of the target nucleic acid and/or its complement. An amplified product, as described herein will generally be double-stranded DNA, although reference can be made to individual strands thereof.

In some embodiments, the methods and compositions described herein relate to multiplex PCR. As used herein, “multiplex PCR” refers to a variant of PCR where simultaneous amplification of more than one target polynucleotide and/or more than one polymorphic variant of a target site locus or target nucleic acid in one reaction vessel and subsequent or concurrent detection of the multiple products can be accomplished by using more than one pair of primers in a set (e.g. at least more than one forward and/or more than one reverse primer). Multiplex amplification can be useful not only for detecting the presence of a plurality of targets but also for the analysis, detection, and/or genotyping of deletions, mutations, and polymorphisms, or for quantitative assays. Multiplex can refer to the detection of between 2-1,000 different target sequences and/or polymorphisms of a target site locus or target nucleic acid in a single reaction. As used herein, multiplex refers to the detection of any range between 2-1.000. e.g., between 5-500, 25-1000, or 10-100 different target sequences and/or polymorphisms of a target site locus or target nucleic acid in a single reaction, etc. By way of non-limiting example, a multiplex PCR reaction as part of a method described herein can affirmatively detect the presence of two or more possible alleles of at least two SNPs at at least two different allelic target site loci in a single reaction. The term “multiplex” as applied to PCR implies that there are primers specific for at least two different target sequences in the same PCR reaction. Thus, a reaction in which there are primer sets specific for two different target sequences is considered a multiplex amplification even if only one (or even none) of the at least two target sequences is actually detected in a given sample. Thus, in some embodiments, multiplex PCR can also refer to reaction containing multiple pairs of primers, wherein the reaction can result in one or multiple specific amplified products when one or multiple targets are present in the reaction.

In some embodiments, the methods and compositions described herein relate to multimodal PCR. As used herein, “multimodal” refers to a variant of multiplex PCR where simultaneous amplification of more than one type or class of target sequence occurs in one reaction vessel. Multimodal amplification can be useful for analysis of deletions, mutations, and polymorphisms in some embodiments. Multimodal can refer to the detection of at least two different types of targets, i.e. 2 different types of targets, 3 different types of targets, 4 different types of targets, or 5 or more different types of targets. By way of non-limiting example, a multimodal PCR reaction can detect the presence of an mRNA and an miRNA in a single reaction, including quantitation of such target, or a multimodal PCR can genotype a SNP and detect an miRNA in the same reaction. By way of further non-limiting example a multimodal PCR amplification can genotype a SNP and detect a deletion at a second target site locus in a single reaction.

Thus, a multimodal reaction can detect, for example, the presence and/or amount of an expressed RNA and a DNA genotype in the same reaction. In some embodiments, the methods and compositions described herein relate to quantitative detection of a target nucleotide, polymorphisms of target nucleotides or polynucleotides, and/or allelic target site loci. In some embodiments, the methods and compositions described herein relate to quantitative multiplex detection of target nucleotides or polynucleotides, polymorphisms of target polynucleotides, and/or allelic target site loci. In some embodiments, the methods and compositions described herein relate to quantitative multimodal detection of a target nucleotide, polymorphisms of target nucleotides, and/or allelic target site loci. In some embodiments, in order to specifically detect RNA or cDNA in the presence of DNA, one or more primers can be intron-spanning primers. In some embodiments, in order to detect short target polynucleotides (e.g. miRNAs or degraded target polynucleotides) as well as longer target polynucleotides (e.g. mRNA or target site loci in genomic DNA), primers for at least the shorter target polynucleotides can comprise tag sequence that results in an amplified product of larger, discrete size than the target sequence. The tags can be designed such that all amplified products in a reaction will be of distinct sizes.

Quantitative aspects can be facilitated, for example, by repeated sampling at any time during or after an amplification reaction, followed by separation and detection of the amplification products. Sampling can, for example, comprise removing an aliquot of the reaction. Sampling can occur, for example, at the end of every cycle, or at the end of every several cycles, e.g. every two cycles, every three cycles, every four cycles etc. While a uniform sample interval will most often be desired, there is no requirement that sampling be performed at uniform intervals. As just one example, the sampling routine can involve sampling after every cycle for the first five cycles, and then sampling after every other cycle or vice versa.

Sampling or dispensing of an aliquot from an amplification reaction can be performed in any of several different general formats. The sampling or removal method can depend on any of a number of factors including, but not limited to, the equipment available, the number of samples to be analyzed, and the timing of detection relative to sample collection (e.g., concurrently vs. sequential). The exact method of removal or extrusion of samples is not necessarily a limitation of the methods described herein. Sampling is preferably performed with an automated device, especially for high throughput applications. Sampling can also be performed using direct electrokinetic or hydrodynamic injection from a PCR reaction into a capillary electrophoretic device. The method of sampling used in the methods is preferably adapted to minimize contamination of the cycling reaction(s), by, for example, using pipetting tips or needles that are either disposed of after a single aliquot is withdrawn, or by using the same tip or needle for dispensing the sample from the same PCR reaction vessel. Methods for simultaneous sampling and detection are known to those skilled in the art (see, e.g., US Patent Application Publication 2004/0166513, incorporated herein by reference).

The amount of nucleic acid and/or volume of an aliquot dispensed at the sampling step can vary, depending, for example, upon the total volume of the amplification reaction, the sensitivity of product detection, and the type of separation used. Amplification volumes can vary from several microliters to several hundred microliters (e.g., 5 μl, 10 μl, 20 μl, 40 μl, 60 μl, 80 μl, 100 μl, 120 μl, 150 μl, or 200 μl or more), preferably in the range of 10-150 μl, more preferably in the range of 10-100 μl. The exact volume of the amplification reaction is not a limitation of the invention. Aliquot volumes can vary from 0.01% to 30% of the reaction mixture. The amplification regimen can be performed on plural independent nucleic acid amplification mixtures, optionally in a multiwell container. The container(s) in which the amplification reaction(s) are preformed is not necessarily a limitation of the methods described herein.

In some embodiments, the methods described herein relate to a PCR amplification regimen comprising at least 2 phases. In some embodiments, the methods described herein relate to a PCR amplification regimen consisting of 2 phases. As used herein, a “phase” of a PCR amplification regimen is a first set of cycles that can be differentiated from a second set of cycles by the sets of cycles necessarily having different annealing temperatures and generally having different numbers of cycles. In some embodiments, all other time and temperature parameters are identical. In some embodiments, additional time and temperature parameters can vary. In some embodiments, the annealing temperature of the first phase is lower than the annealing temperature of the second phase. As used herein, a “set of cycles” refers to one or more cycles of a PCR amplification regimen which have identical temperature parameters.

In some embodiments, the first phase annealing temperature is lower than the second phase annealing temperature. In some embodiments, the first annealing temperature is lower than the second annealing temperature by at least 2° C., i.e. by 2° C., by 3° C., by 4° C., by 5° C., by 6° C., by 8° C., by 10° C., or more. In some embodiments, the first annealing temperature is lower than the second annealing temperature by at least 4° C.

In some embodiments, each phase of the PCR amplification regimen comprises at least 1 cycle, e.g. 1 cycle, 2 cycles, 3 cycles, 4 cycles, 5 cycle, 10 cycles, 20 cycles or more cycles. In some embodiments, the first phase of the PCR amplification regimen comprises fewer than 10 cycles, e.g. 10 cycles, 9 cycles, 8 cycles, 7 cycles, 6 cycles, 5 cycles, 4 cycles, 3 cycles, 2 cycles or 1 cycle. As noted above, keeping the number of cycles in the first phase to a minimum can avoid or at least mitigate the impact of mispriming to non-target sequences in the initial nucleic acid sample. In some embodiments, the first phase of the PCR amplification regimen comprises from 3 to 5 cycles. In some embodiments, the first phase of the PCR amplification regimen comprises 3 cycles. In some embodiments, the first phase of the PCR amplification regimen comprises 4 cycles. In some embodiments, the first phase of the PCR amplification regimen comprises 5 cycles.

In some embodiments, the second phase of the PCR amplification regimen comprises at least 5 cycles, e.g. 5 cycles, 10 cycles, 15 cycles, 20 cycles, 25 cycles, 30 cycles or more cycles. In the second phase, the higher annealing temperature substantially prevents priming from non-target sequences in the initial nucleic acid sample, thereby favoring amplification of only target polynucleotides amplified in the first phase of the regimen. Thus, this second phase can include more cycles, substantially without risk of increasing background. This approach, coupled with rules for differences in 5′ tail sequences can substantially decrease background in PCR reactions in general, and in SNP genotyping reactions in particular. In some embodiments, the entire PCR amplification regimen comprises at least 10 total cycles, e.g. 10 cycles, 15 cycles, 20 cycles, 25 cycles, 30 cycles, 35 cycles, 40 cycles or more.

PCR Targets

In some embodiments, the methods and compositions described herein relate to detecting the presence of a target nucleic acid and/or polynucleotide. A target nucleic acid can be an RNA or a DNA. A target nucleic acid can be a double-stranded (ds) nucleic acid or a single-stranded (ss) nucleic acid, e.g. a dsRNA, a ssRNA, a dsDNA, or a ssDNA. As noted above, it is specifically contemplated that methods described herein permit the detection and/or quantitation of more than one of these types of target in the same reaction, i.e. multimodal amplification and detection. Non-limiting examples of target nucleic acids include a nucleic acid sequence, a nucleic acid sequence comprising a mutation, a nucleic acid sequence comprising a deletion, a nucleic acid sequence comprising an insertion, a sequence variant, an allele, a polymorphism, a SNP, a microRNA, a protein coding RNA, a non-protein coding RNA, an mRNA, a nucleic acid from a pathogen (e.g. a bacterium, a virus, or a parasite), a nucleic acid associated with a disease or a likelihood of having or developing a disease (e.g. a marker gene, a polymorphism associated with a disease or a likelihood of having or developing a disease, or an RNA, the expression of which is associated with a disease or a likelihood of having or developing a disease).

In some embodiments, the methods and compositions described herein relate to detecting the presence of one or more polymorphisms of a target nucleic acid and/or polynucleotide. As used herein, the term “polymorphism” refers to a nucleic acid sequence variation. When compared to a predominantly occurring natural sequence, a polymorphism can be present at a frequency of greater than 0.01%, 0.1%, 1% or greater in a population. As used herein, a polymorphism can be a substitution, insertion, deletion, duplication, or rearrangement. A polymorphism, including a single nucleotide polymorphism (SNP), can be phenotypically neutral or can have an associated variant phenotype that distinguishes it from that exhibited by the predominant sequence at that locus. As used herein, “neutral polymorphism” refers to a polymorphism in which the sequence variation does not alter gene function, and “mutation” or “functional polymorphism” refers to a sequence variation which does alter gene function, and which thus has an associated phenotype. Polymorphisms of a locus occurring in a population are referred to as alleles. When referring to the genotype of an individual with regard to a specific locus at which two or more alleles occur within a population, the “predominant allele” is that which occurs most frequently in the population in question (i.e., when there are two alleles, the allele that occurs in greater than 50% of the population is the predominant allele; when there are more than two alleles, the “predominant allele” is that which occurs in the subject population at the highest frequency, e.g., at least 5% higher frequency, relative to the other alleles at that site). The term “variant allele” is used to refer to the allele or alleles occurring less frequently than the predominant allele in that population (e.g., when there are two alleles, the variant allele is that which occurs in less than 50% of the subject population; when there are more than two alleles, the variant alleles are all of those that occur less frequently, e.g., at least 5% less frequently, than the predominant allele).

As used herein, a “single nucleotide polymorphism” or “SNP” refers to nucleic acid sequence variation at a single nucleotide residue, including a single nucleotide deletion, insertion, or base change or substitution. SNPs can be allelic. Some SNPs have defined phenotypes, e.g. disease phenotypes, while others have no known associated phenotype. SNP detection methods, described herein can be used for the prediction of phenotypic characterisitics, e.g. prediction of responsiveness or sensitivity to drugs. In this regard, SNP genotyping as described herein and known in the art is not necessarily diagnostic of disease or susceptibility to disease.

In some embodiments, the methods and compositions described herein relate to determining the genotype of a nucleic acid sample at one or more allelic target site loci. As used herein, the term “target site locus” refers to a genetic locus which is to be genotyped. A target site locus generally has a wild-type allele and at least one variant allele. A target site locus can comprise the nucleotide position(s) which vary between alleles of the target site locus. A target site locus is not limited to only the particular nucleotide position(s) that vary between alleles, but can comprise the sequences flanking the variable nucleotide positions. In general, a “target site locus” is a sequence at least big enough to be PCR amplified using a primer set as described herein. Generally, a target site locus can be less than 5 kb in size. Two target site loci can overlap, e.g. as in the case of two SNPs located 10 bp (or fewer) apart. In this instance, each SNP will be flanked by a target site locus, which will have considerable overlap with the target site locus which flanks the other SNP. The methods described herein are particularly well adapted for genotyping SNPs that fall closely together. When used in reference to a SNP, the “target site” is the actual nucleotide that varies.

In some embodiments, the methods and compositions described herein relate to determining the genotype of a nucleic acid sample at one or more target site loci in the same reaction. In some embodiments, the methods and compositions described herein relate to a set of oligonucleotide primers specific for each target site locus. The set of oligonucleotide primers can comprise primers specific for one or more alleles of the locus, e.g. for the predominant allele, the wild-type allele, the variant allele(s), and/or the mutant allele(s). The set of oligonucleotide primers can comprise 2 or more primers, i.e. 2 primers, 3 primers, 4 primers, 5 primer, 6 primers, 7 primers, or more primers.

As noted, in some embodiments, a target site locus comprises a SNP. Four alleles of a SNP locus are possible, although SNPs that vary only between two nucleotides at the target site are not uncommon. In some embodiments, the methods and compositions described herein relate to a set of primers that can detect a single allele of a SNP locus. In some embodiments, the methods and compositions described herein relate to a set of primers that can detect two alleles of a SNP locus (i.e. the methods and compositons can relate to an assay that permits the affirmative detection of two SNP alleles, or “biphasic” genotyping of that SNP). In some embodiments, the methods and compositions described herein relate to a set of primers that can detect three alleles of a SNP locus (i.e. the methods and compositons can relate to an assay that permits the affirmative detection of three SNP alleles, or “triphasic” genotyping of that SNP). In some embodiments, the methods and compositions described herein relate to an assay that permits affirmative detection of four alleles of a SNP locus (i.e. the methods and compositons can relate to a multiplex detection of four SNP alleles, or “quaduphasic” genotyping of that SNP). In some embodiments, the predominant and/or wild-type allele of a SNP is detected. In some embodiments, the predominant and/or wild-type allele of a SNP is not detected. By “affirmatively detected” is meant that the assay permits the amplification of that specific allele. An alternative to affirmative detection can be used, for example, when there are only two possibilities known to exist at the SNP site. In this instance, the assay can be designed such that one of the two variants is amplified, and the other is not; the assay can affirmatively detect that amplified variant and passively detect the other, i.e. the lack of a product means the other allele or variant is present.

Samples for Analysis

The methods and compositions described herein relate to the detection of the presence of a target nucleic acid in a sample. A sample useful herein will comprise nucleic acids. In some embodiments, a sample can further comprise proteins, cells, fluids, biological fluids, preservatives, and/or other substances. In some embodiments, a sample can be obtained from a subject. In some embodiments, a sample can be a biological sample obtained from the subject. In some embodiments a sample can be a diagnostic sample obtained from a subject. By way of non-limiting example, a sample can be a cheek swab, blood, serum, plasma, sputum, cerebrospinal fluid, urine, tears, alveolar isolates, pleural fluid, pericardial fluid, cyst fluid, tumor tissue, tissue, a biopsy, saliva, an aspirate, or combinations thereof. In some embodiments, a sample can be obtained by resection or biopsy.

In some embodiments, the sample comprises microorganisms that are a subject of the analysis. In some embodiments, the sample comprises a portion or aliquot of a culture of microorganisms.

In some embodiments, the sample is a clarified fluid sample, for example, by centrifugation. In some embodiments, the sample is clarified by low-speed centrifugation (e.g. 3,000×g or less) and collection of the supernatant comprising the clarified fluid sample.

In some embodiments, the sample can be freshly collected. In some embodiments, the sample can be stored prior to being used in the methods and compositions described herein. In some embodiments, the sample is an untreated sample. As used herein, “untreated sample” refers to a biological sample that has not had any prior sample pre-treatment except for dilution and/or suspension in a solution.

In some embodiments, a sample can be obtained from a subject and preserved or processed prior to being utilized in the methods and compositions described herein. By way of non-limiting example, a sample can be embedded in paraffin wax, refrigerated, or frozen. A frozen sample can be thawed before determining the presence of a nucleic acid according to the methods and compositions described herein. In some embodiments, the sample can be a processed or treated sample. Exemplary methods for treating or processing a sample include, but are not limited to, centrifugation, filtration, sonication, homogenization, heating, freezing and thawing, contacting with a preservative (e.g. anti-coagulant or nuclease inhibitor) and any combination thereof. In some embodiments, the sample can be treated with a chemical and/or biological reagent. Chemical and/or biological reagents can be employed to protect and/or maintain the stability of the sample or nucleic acid comprised by the sample during processing and/or storage. In addition, or alternatively, chemical and/or biological reagents can be employed to release nucleic acids from other components of the sample. By way of non-limiting example, a blood sample can be treated with an anti-coagulant prior to being utilized in the methods and compositions described herein. The skilled artisan is well aware of methods and processes for processing, preservation, or treatment of samples for nucleic acid analysis.

In some embodiments, the nucleic acid present in a sample is isolated, enriched, or purified prior to being utilized in the methods and compositions described herein. Methods of isolating, enriching, or purifying nucleic acids from a sample are well known to one of ordinary skill in the art. By way of non-limiting example, kits for isolation of genomic DNA from various sample types are commercially available (e.g. Catalog Nos. 51104, 51304, 56504, and 56404; Qiagen; Germantown, Md.).

The terms “subject” and “individual” are used interchangeably herein, and refer to an organism from which a sample is obtained. A subject can be any organism for which it is desired to determine the presence of a nucleic acid in the organism or one or more cells comprising or contained within that organism. As used herein, a “subject” can mean an organism, e.g. a bacterium, a parasite, a plant, or an animal. In some embodiments, a subject can be a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Individual or subject includes any subset of the foregoing, e.g., all of the above.

Primers

In various embodiments, the methods and compositions described herein relate to performing a PCR amplification regimen with at least one set of oligonucleotide primers specific for each target polynucleotide, target polymorphism, and/or allelic target site locus. As used herein, “primer” refers to a DNA or RNA polynucleotide molecule or an analog thereof capable of specifically annealing to a polynucleotide template and providing a 3′ end that serves as a substrate for a template-dependent polymerase to produce an extension product which is complementary to the polynucleotide template. The conditions for initiation and extension usually include the presence of at least one, but more preferably all four different deoxyribonucleoside triphosphates and a polymerization-inducing agent such as DNA polymerase or reverse transcriptase, in a suitable buffer (in this context “buffer” includes solvents (generally aqueous) plus necessary cofactors and reagents which affect pH, ionic strength, etc.) and at a suitable temperature. A primer useful in the methods described herein is generally single-stranded, and a primer and its complement can anneal to form a double-stranded polynucleotide. Primers according to the methods and compositions described herein can be less than or equal to 300 nucleotides in length, e.g., less than or equal to 300, or 250, or 200, or 150, or 100, or 90, or 80, or 70, or 60, or 50, or 40, and preferably 30 or fewer, or 20 or fewer, or 15 or fewer, but at least 10 nucleotides in length.

As used herein, the term “set” means a group of nucleic acid samples, primers or other entities. A set will comprise a known number of, and at least two of such entities. A set of primers comprises at least one forward primer and at least one reverse primer specific for a target site locus and/or target polynucleotide nucleotide.

Thus, as used herein, “set of oligonucleotide primers” refers to a group of at least two primers, including a forward primer and a reverse primer, one of which anneals to a first strand of a target nucleic acid and the other of which anneals to a complement of the first strand. In some embodiments, the primers of a first subset (e.g., forward primers) can anneal to a first strand of a target nucleic acid sequence and the primers of the second subset (e.g., reverse primers), can anneal to the complement of that strand. The orientation of the primers when annealed to the target and/or its complement can be such that nucleic acid synthesis proceeding from primer extension of a primer of one subset would produce a nucleic acid sequence that is complementary to at least one region of a primer of the second subset. The “first strand” of a nucleic acid target and/or sequence can be either strand of a double-stranded nucleic acid comprising the sequence of the target nucleotide and/or target site locus, but once chosen, defines its complement as the second strand. Thus, as used herein, a “forward primer” is a primer which anneals to a first strand of a nucleic acid target, while a “reverse primer” of the same set is a primer which anneals to the complement of the first strand of the nucleic acid target.

As used herein, the term “subset” means a group comprised by a set as defined herein, wherein the subset group is less than every member of the set. A subset as used herein can consist of a single entity from within a set. A “subset of primers” specific for a target nucleic acid and/or a polymorphic target site locus refers to a group of primers specific for the same target nucleic acid and/or polymorphic target site locus which anneal, for example, to a first strand of the target nucleic acid and not to second strand of the target nucleic acid. A subset of primers comprises, for example at least one forward primer specific for a target site locus and/or target nucleotide sequence, e.g. one forward primer, two forward primers, three forward primers, or more forward primers specific for a target site locus and/or target nucleotide sequence. Alternatively, a subset of primers comprises, for example at least one reverse primer specific for a target site locus and/or target nucleotide sequence, e.g. one reverse primer, two reverse primers, three reverse primers, or more reverse primers specific for a target site locus and/or target nucleotide sequence.

As used herein, “specific” when used in the context of a primer specific for a target nucleic acid refers to a level of complementarity between the primer and the target such that there exists an annealing temperature at which the primer will anneal to and mediate amplification of the target nucleic acid and will not anneal to or mediate amplification of non-target sequences present in a sample.

Methods of making primers are well known in the art, and numerous commercial sources offer oligonucleotide synthesis services suitable for providing primers according to the methods and compositions described herein, e.g. INVITROGEN™ Custom DNA Oligos; Life Technologies; Grand Island, N.Y. or custom DNA Oligos from IDT; Coralville, Iowa).

Dual Domain Primers

In various embodiments, the methods and compositions described herein relate to a set of oligonucleotides comprising at least one dual domain primer. As discussed above, dual domain primers permit the amplification and detection of specific sequences while avoiding at least two sources of error in PCR detection and quantitation. As used herein, a “dual domain primer” refers to a primer having a 5′ tail region, and a 3′ core region complementary to either a first or second strand of a target polynucleotide and one or more 3′ terminal nucleotides complementary to one of the variant nucleotides occurring at a target site locus. A “truncated” dual domain primer does not have a 3′ terminal nucleotide specific for one allele of a SNP. The term “truncated” as used in this context does not imply anything regarding the length of the primer, but is intended to distinguish, for example, primers not designed to specifically detect one SNP allele. In some embodiments, a truncated dual domain primer can be part of a set of primers also comprising at least one dual domain primer in order to detect and amplify at least one allele of a SNP. In some embodiments, one or more truncated dual domain primers can be used in a primer set to detect target polynucleotides other than SNP alleles, e.g. mRNAs. The dual domain and truncated dual domain primers, as well as their roles and uses, are discussed further below. Specific characteristics or rules with respect to the design and function of the 5′ tail and 3′ core region of a dual domain primer and/or truncated dual domain primer are discussed in the following.

5′ Tail Region

The 5′ tail region of a dual domain primer and/or truncated dual domain primer is, necessarily, 5′ of the 3′ core region and, as a rule, does not have any homology to the target sequence. This can be determined by creating an alignment of the sequence of the primer to the nucleic acid target sequence (or a complement thereof depending upon the orientation of the primer) via the 3′ core region of the dual domain primer and/or truncated dual domain primer. At each position of the 5′ tail region, the nucleotide base present at that position should not be the same as the nucleotide base found in the nucleic acid target sequence at the corresponding position, i.e. it should not be homologous. Alignments necessary to confirm lack of homology for a 5′ tail sequence can be done by eye. However, if necessary, Ooe of ordinary skill in the art is aware of software available for creating alignments of nucleic acid sequences, e.g. BLAST or CLUSTALW2. Default parameters are generally sufficient for the alignments necessary to establish lack of target sequence homology.

In addition to lacking homology to the target sequence, as a rule, the sequence of the 5′ tail region of a dual domain primer differs from the sequence of the 5′ tail region of other dual domain and/or truncated dual domain primers in the same set, i.e. it is not identical to the 5′ tail region of other dual domain primers and/or truncated dual domain primers in the same set. In some embodiments, the sequence of a 5′ tail region of a dual domain primer and/or truncated dual domain primer has 6 or fewer contiguous homologous bases relative to the sequence of the 5′ tail region of other dual domain primers and/or truncated dual domain primers in the same set, i.e. it has no more than 6 contiguous homologous bases, it has no more than 5 contiguous homologous bases, it has no more than 4 contiguous homologous bases, it has no more than 3 contiguous homologous bases, it has no more than 2 contiguous homologous bases, it has only 1 homologous base, or it has no homologous bases. As used herein, “contiguous bases” refers to bases which form an uninterrupted sequence of bases within a nucleic acid. For example, two bases are contiguous if one is immediately 5′ or 3′ of the other in a nucleic acid sequence, i.e. there are no intervening bases between them. Examples of tail sequences suitable for use in the same set or subset of primers are seen in Tables 2 and 6.

In some embodiments, in the case of 2 dual domain primers and/or truncated dual domain primers, each of which is comprised by a different set of oligonucleotides, (e.g. a set of oligonucleotide primers specific for a different target locus) the 2 primers can have 5′ tail regions that have 1 or more homologous bases, i.e. they have 1 homologous base, 2 homologous bases, 3 homologous bases, 4 homologous bases, 5 homologous bases, 6 homologous bases or more homologous bases. In some embodiments, in the case of 2 dual domain primers and/or truncated dual domain primers, each of which is comprised by a different set of oligonucleotides, the 2 primers can have 5′ tail regions which are identical.

In some embodiments, the 5′ tail region of a dual domain primer or truncated dual domain primer as described herein can be from 1 to 200 nucleotides or longer in length, e.g. it can be 1 nucleotide, 2 nucleotides, 3 nucleotides, 4 nucleotides, 5 nucleotides, 6 nucleotides, 7 nucleotides, 8 nucleotides, 9 nucleotides, 10 nucleotides, 12 nucleotides, 14 nucleotides, 16 nucleotides, 18 nucleotides, 20 nucleotides, 30 nucleotides, 40 nucleotides, 50 nucleotides, 60 nucleotides, 70 nucleotides, 80 nucleotides, 90 nucleotides, 100 nucleotides in length, or any other length in between these values, or longer. In theory, a 5′ tail could be much longer. However, practically speaking, primers are generally 300 nucleotides or less in length, preferably shorter. It should be kept in mind that the longer the tail, the more difficult it can become to exclude regions of homology to one or more targets in a multiplex reaction and the more likely it is that another primer can hybridize to it. As such, 5′ tail sequences of 200 nucleotides or less are preferred. While the 5′ tail can be as little as 1 nucleotide in length, this would only be acceptable if the 1 nucleotide difference were sufficient to increase the Tₘ as required for a 5′ tail to be functional as described herein. Small differences in tail length and composition will necessarily have the greatest impact on Tₘ when the 3′ core region is relatively short, as the impact of any single nucleotide on Tₘ diminishes the longer the polynucleotide becomes. As noted above, a critical aspect of the 5′ tail region is that it increases the Tm for the dual domain primer, or truncated dual domain primer, relative to a primer that hybridizes to the target region alone, i.e. a primer with 3′ core sequence alone. Thus, the choice of length and composition of the 5′ tail region is driven substantially by this functional requirement. As used herein, the term “increases the Tₘ” means that a change to a primer, e.g. addition of a 5′ tail sequence, is sufficient to raise the Tₘ of the resulting primer, under salt and buffer conditions suitable for a PCR reaction mixture such that the altered primer can substantially anneal to its target sequence at a temperature at which the original primer can no longer substantially anneal to its target sequence.

In some embodiments, the 5′ tail region of a dual domain primer or truncated dual domain primer as described herein can be from 1 to 200 nucleotides in length. Preferably, the 5′ tail region of the dual domain or truncated dual domain primer can be 150 nucleotides or less in length. In some embodiments, the 5′ tail region of a dual domain primer or truncated dual domain primer as described herein can be from 4 to 150 nucleotides in length. In some embodiments, the 5′ tail region of a dual domain primer or truncated dual domain primer as described herein can be from 1 to 10 nucleotides in length. In some embodiments, the 5′ tail region of a dual domain primer or truncated dual domain primer as described herein can be at least 2 nucleotides in length, e.g. at least 2 nucleotides in length, at least 3 nucleotides in length, at least 4 nucleotides in length, at least 5 nucleotides in length, at least 6 nucleotides in length, at least 7 nucleotides in length, at least 8 nucleotides in length, at least 9 nucleotides in length or longer.

Exemplary examples of 5′ tail region sequences are provided as SEQ ID NOs: 210-2709. SEQ ID NOs: 210-459 have a GC content of 20%. SEQ ID NOs: 460-709 have a GC content of 30%. SEQ ID NOs: 709-959 have a GC content of 40%. SEQ ID NOs: 960-1209 have a GC content of 50%. SEQ ID NOs: 1210-1459 have a GC content of 60%. SEQ ID NOs: 1460-1709 have a GC content of 25%. SEQ ID NOs: 1710-1959 have a GC content of 35%. SEQ ID NOs: 1960-2209 have a GC content of 45%. SEQ ID NOs: 2210-2459 have a GC content of 55%. SEQ ID NOs: 2460-2709 have a GC content of 65%. Combinations of any of SEQ ID NOs: 210-2709 can be used in a single multi-modal assay as described elsewhere herein and can be selected, e.g. by Tm in order to provide for dual domain primers with similar Tm's depending upon the 3′ core regions which they will be linked to in order to form complete dual domain primers.

Accordingly, in some embodiments, described herein is a composition for determining the presence of one or more target polynucleotides, comprising; at least one set of oligonucleotide primers specific for each target polynucleotide; wherein each said set of oligonucleotide primers comprises a first subset of at least one truncated dual domain forward primer and a second subset of at least one reverse primer; wherein each truncated dual domain primer of a set comprises a 5′ tail region that differs from the 5′ tail region on other truncated dual domain primers in the set and a 3′ core region complementary to a sequence on one strand of a double-stranded nucleic acid comprising said target; wherein for each truncated dual domain primer, the 3′ core substantially anneals to its complementary target site sequence at a first annealing temperature, and the sequence comprised by the 5′ tail and 3′ core region substantially anneals to its complement at a second annealing temperature, the second annealing temperature being higher than the first annealing temperature, such that at said second annealing temperature said 3′ core of said truncated dual domain primer cannot substantially anneal to a template molecule that does not also have the complement of the primer's 5′ tail sequence; wherein for each member of said truncated dual domain primer set: the 5′ tail sequence does not have any homology to the target sequence; and the 5′ tail sequence has 6 or fewer contiguous homologous bases relative to the other 5′ tail sequences of the truncated dual domain primer set and wherein each 5′ tail sequence is selected from SEQ ID NOs: 210-2709 or sequences having no more than four mutations, insertions, or deletions (e.g. four mutations, insertions, or deletions, three mutations, insertions, or deletions, two mutations, insertions, or deletions, or one mutation, insertion, or deletion) relative to one of SEQ ID NOs: 210-2709. In the context of this paragraph, an insertion refers to the insertion of only one nucleotide, e.g. 2 insertions, even at the same site, refers to the addition of only 2 nucleotides. Similarly, in the context of this paragraph, a deletion refers to the deletion of only one nucleotide, e.g. 2 deletions, even at the same site, refers to the deletion of only 2 nucleotides.

3′ Core Region

As used herein, a “3′ core region” of a dual domain primer refers to a region of a primer located 3′ of the 5′ tail region and which is complementary to either a first or second strand of the target polynucleotide. In some embodiments, the dual domain primer is to be used for genotyping or determining the presence of alleles and/or variants of a target site locus; in those instances, the 3′ core region can be designed to flank the portion of the target site locus which varies between alleles and/or variants. A set of dual domain primers for genotyping a given variable site will generally have one dual domain primer for each nucleotide occurring at that variable site, each of the different primers having a 3′ terminal nucleotide complementary to only one of the nucleotides occurring at that site.

In some embodiments, the methods and compositions described herein relate to a set of oligonucleotides comprising at least one truncated dual domain primer. As used herein, a “truncated dual domain primer” refers to a primer having a) a 5′ tail region and b) a 3′ core region complementary to either a first or second strand of the target polynucleotide. A truncated dual domain primer lacks the 3′ terminal nucleotides of a dual domain primer which are specific to a particular allele of a polymorphism. As with other dual domain primers, the 5′ tail region of a truncated dual domain primer differs from the 5′ tail region of other dual domain primers and/or truncated dual domain primers in the same set.

The 3′ core region of a dual domain primer or truncated dual domain primer as described herein can be from 10 to 40 nucleotides in length.

In some embodiments, a set of oligonucleotide primers comprises a subset of forward primers comprising two or more dual domain or truncated dual domain primers, each of which is designed to specifically anneal to and amplify an allele of the same target locus. The set of primers can further comprise a single reverse primer, that reverse primer comprising a truncated dual domain primer, which will act in concert with each forward primer of the oligonucleotide primer set to produce distinguishable amplification products during a PCR amplification regimen (see for Example, the Kras primer set of Table 2). Alternatively, the subset can comprise two or more dual domain or truncated dual domain reverse primers, each designed to anneal to and amplify an allele of the same target locus, and a single forward primer.

In some embodiments, the methods and compositions described herein relate to a set of oligonucleotides comprising at least one amplifying primer. As used herein, an “amplifying primer” refers to a primer having a region complementary to either a first strand of the target nucleic acid or the complement of the first strand. As contrasted with a dual domain or truncated dual domain primer, an amplifying primer does not comprise a 5′ tail region, nor does it comprise nucleotides complementary to only one allele or variant of a target nucleic acid.

In some embodiments, each set of oligonucleotide primers can comprise 2 or more primers, e.g. 2 primers, 3 primers, 4 primers, 5 primers, 6 primers or more primers. In some embodiments, each set of oligonucleotide primers can comprise at least n+1 primers, wherein n is the number of polymorphisms and or alleles of the target polynucleotide which are to be detected.

In some embodiments, the Tₘ's for the 3′ core region of two or more primers of a set of primers can vary by no more than 8° C., e.g., by 8° C. or less, by 7° C. or less, by 6° C. or less, by 5° C. or less, by 4° C. or less, by 3° C. or less, by 2° C. or less, by 1° C. or less, or they can be the same.

In some embodiments, the Tₘ's for the 3′ core region of two or more primers present in the same reaction can vary by no more than 8° C. e.g., by 8° C. or less, by 7° C. or less, by 6° C. or less, by 5° C. or less, by 4° C. or less, by 3° C. or less, by 2° C. or less, by 1° C. or less, or they can be the same.

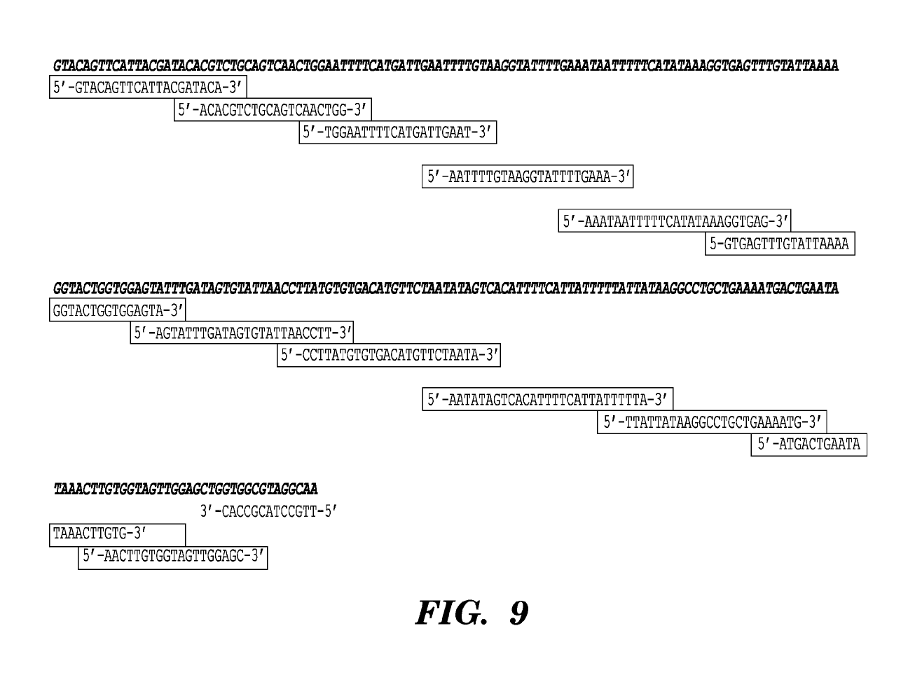

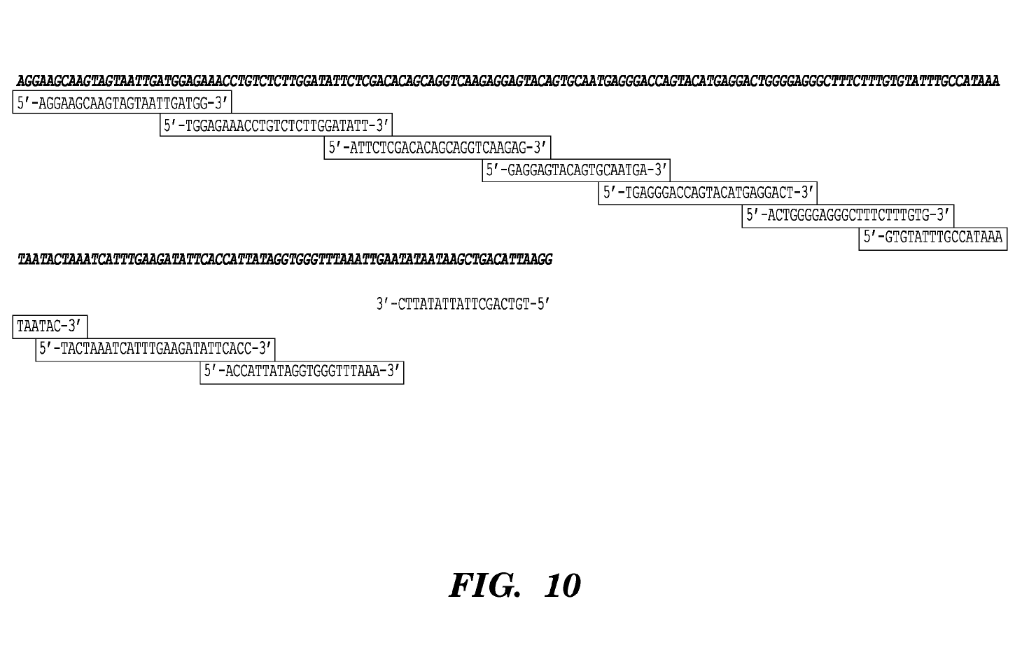

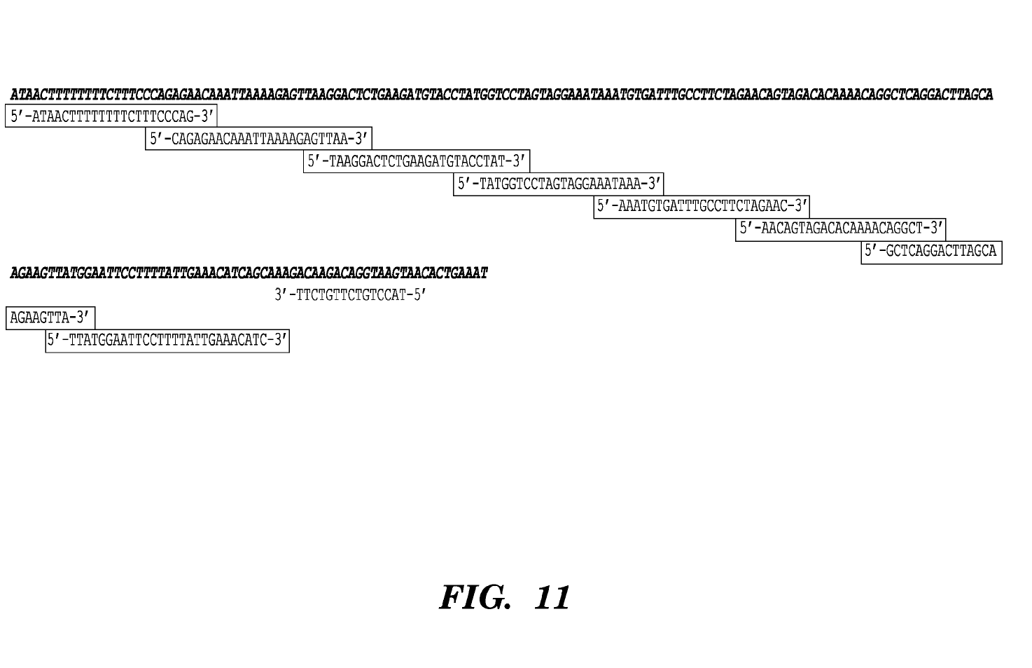

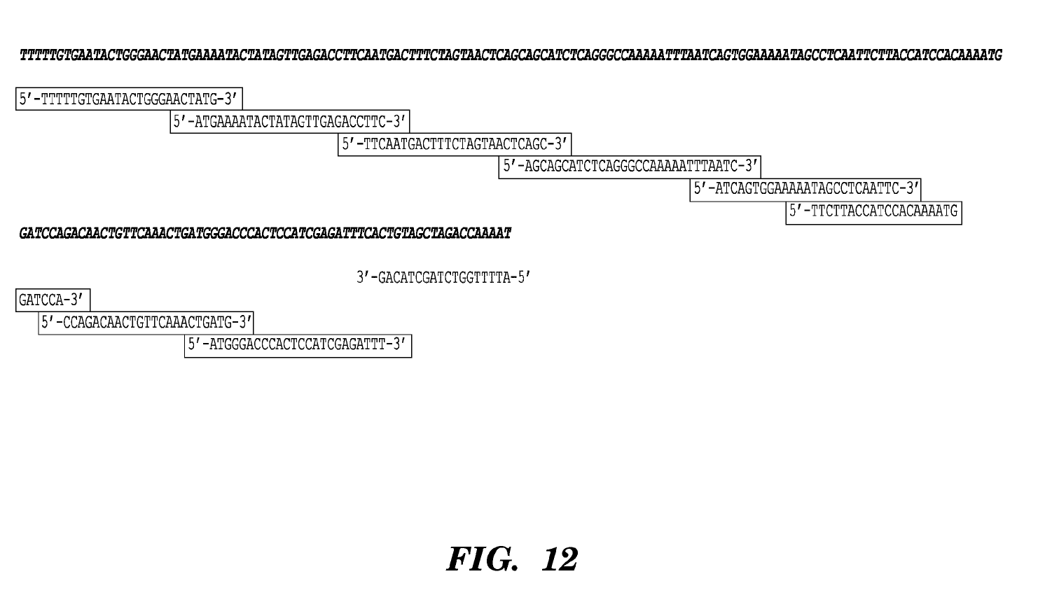

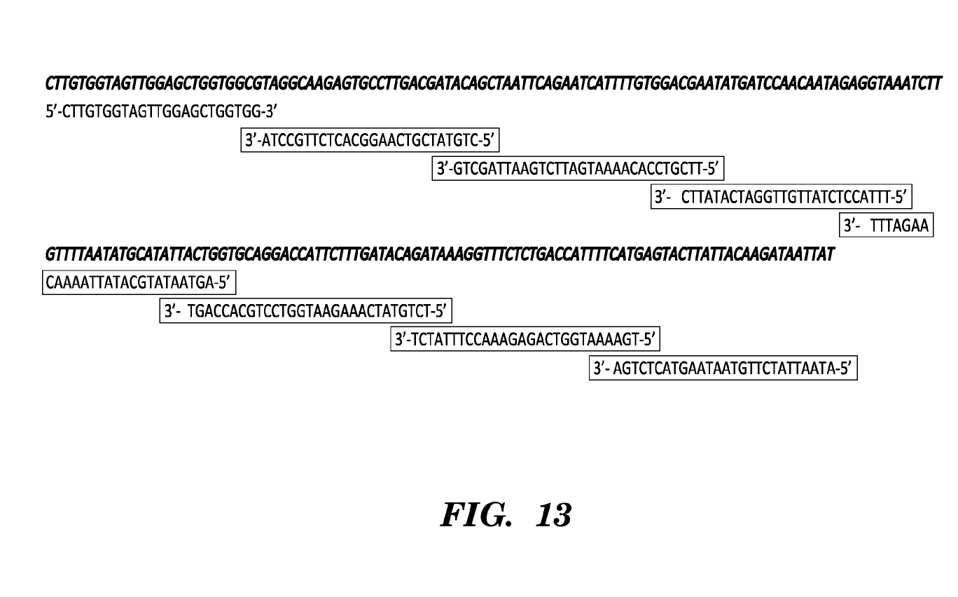

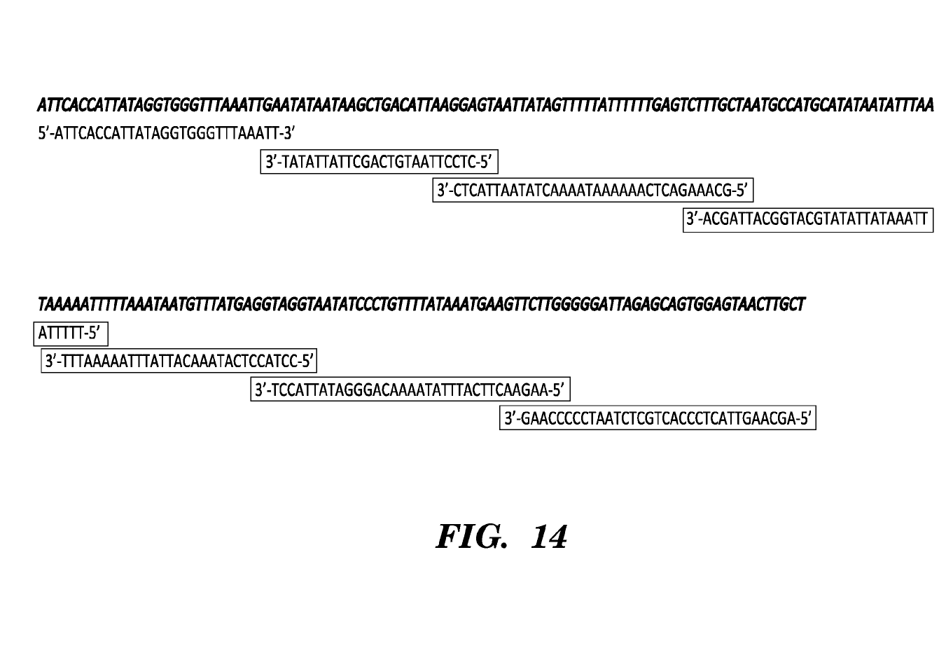

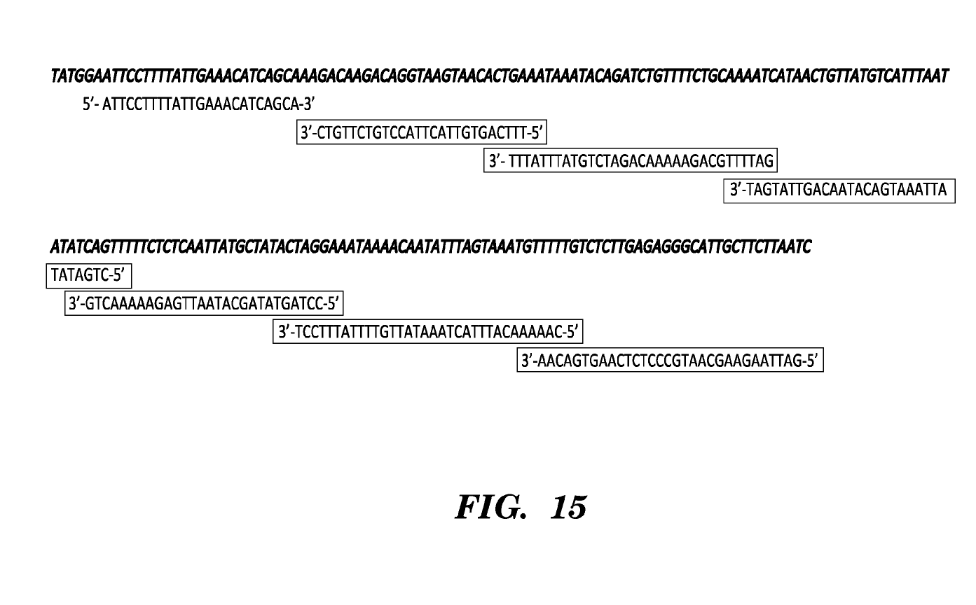

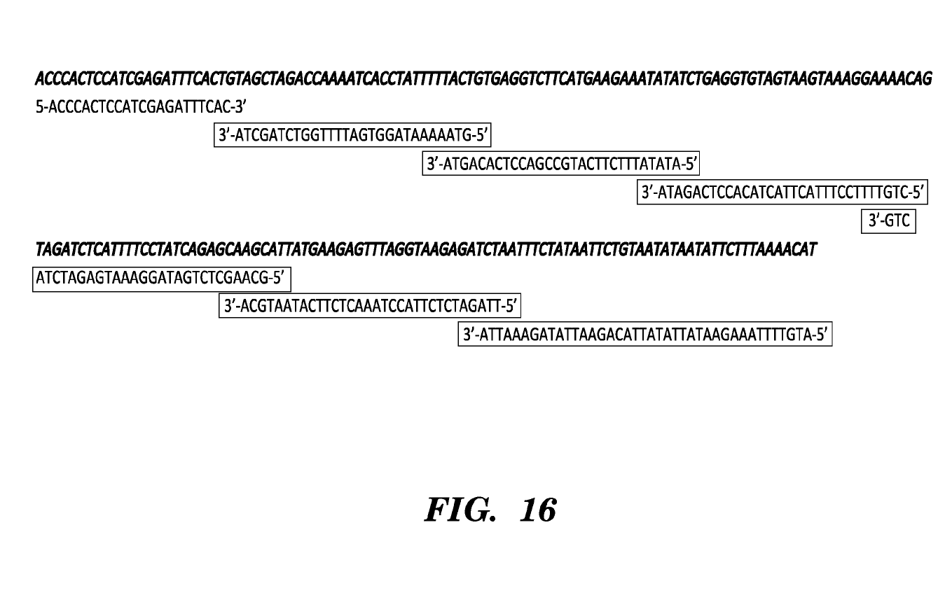

FIGS. 9-16 depict exemplary 3′ core sequences for detecting SNPs in KRAS and BRAF, as indicated in the descriptions of the drawings (e.g. SEQ ID NOs; 86, 100, 111, 121, 131, 140, 148, and 156). Also provided in FIGS. 9-16 are a plurality of reverse primers each of which can be used with a dual domain primer(s) comprising the indicated 3′ core sequence in a PCR reaction.

In some embodiments, a dual domain primer as described herein can comprise any of SEQ ID NOs: 86, 100, 111, 121, 131, 140, 148, and 156 as the 3′ core sequence and any of SEQ ID NOs: 210-2709 as the 5′ tail sequence. In some embodiments, a dual domain primer as described herein can comprise a 3′ core having the sequence of any of SEQ ID NOs: 86, 100, 111, 121, 131, 140, 148 and 156 with one or more substitutions, insertions, or deletions (e.g. one substitution, two substitutions, three substitutions, four substitutions, or more) and a 5′ tail having the sequence of any of SEQ ID NOs: 210-2709 with one or more substitutions, insertions, or deletions (e.g. one substitution, two substitutions, three substitutions, four substitutions, or more). In some embodiments, a dual domain primer as described herein can comprise a 3′ core having the sequence of any of SEQ ID NOs: 86, 100, 111, 121, 131, 140, 148 and 156 with no more than three substitutions, insertions, or deletions (e.g. one substitution, two substitutions, three substitutions, four substitutions, or more) and a 5′ tail having the sequence of any of SEQ ID NOs: 210-2709 with one or more substitutions, insertions, or deletions (e.g. one substitution, two substitutions, three substitutions, four substitutions, or more), but at least 90% identity to one of SEQ ID NOs. 210-2709. A 3′ core sequence bearing substitutions, insertions, or deletions relative to one of SEQ ID Nos: 86, 100, 111, 121, 131, 140, 148 and 156 must retain the ability to specifically hybridize to the complement of SEQ ID NOs: 86, 100, 111, 121, 131, 140, 148 and 156 under initial reaction phase annealing conditions as described herein. In some embodiments, a dual domain primer as described herein can comprise a 3′ core having the sequence of any of SEQ ID NOs: 86, 100, 111, 121, 131, 140, 148 with no more than four substitutions, insertions, or deletions (e.g. one substitution, two substitutions, three substitutions, or four substitutions) and a 5′ tail having the sequence of any of SEQ ID NOs: 210-2709 with no more than four substitutions, insertions, or deletions (e.g. one substitution, two substitutions, three substitutions, or four substitutions). In the context of this paragraph, an insertion refers to the insertion of only one nucleotide, e.g. 2 insertions, even at the same site, refers to the addition of only 2 nucleotides. Similarly, in the context of this paragraph, a deletion refers to the deletion of only one nucleotide, e.g. 2 deletions, even at the same site, refers to the deletion of only 2 nucleotides.

Sequences for use as 3′ core sequences can be generated by selection of any sequence which can specifically anneal to a target (e.g. which is identical to or complementary to either strand of a double-stranded target nucleic acid molecule) by, e.g., selecting a sequence of 10-40 nucleotides in length from the target sequence (e.g. the Kras genes) and can be made into dual domain primers by appending at least a 5′ tail satisfying the requirements set out herein for dual domain primers. In some embodiments, 3′ core sequences can be identified by a method in which a “window” or “mask” of a given size (as a non-limiting example, 40 nucleotides) is literally or figuratively (including, e.g., in silico) placed on the target sequence to identify sequences in the size range that may serve as target sequences. By moving the sequence “window” progressively one nucleotide upstream or downstream of an initial target sequence location, the next potential target sequence can be identified, until the complete set of possible sequences is identified for any given target size selected. This process, coupled with systematic synthesis can identify sequences that can be tested using assay as described herein or as known in the art, thereby establishing those that are optimal for a given gene target. As a non-limiting example, SEQ ID NOs: 2710-93981 are 40-mer sequences obtained from the genomic sequence of K-ras by the use of such a “window” technique, and can be used as 3′ core sequences in the methods and compositions described herein. One of ordinary skill in the art can immediately envision that shorter 3′ core sequences (e.g. 10-39 nucleotides in length) can be obtained from any of SEQ ID NOs: 2710-93981. In some embodiments, a dual domain primer as described herein can comprise a 3′ core sequence having the sequence of any of SEQ ID NOs: 2710-93981 (or a 10-39 nucleotide fragment thereof) and a 5′ tail having the sequence of any of SEQ ID NOs 210-2709.

It is noted that the approach set out herein is effective for any multiplex PCR requiring high sensitivity, i.e. requiring reliable distinctions to be made between closely related or highly similar target sequences.

In some embodiments, the technology described herein relates to a composition according to any of the aspects described herein in which the set of primers comprises at least one primer (e.g., one primer, two primers, three primers, four primers, five primers, six primers, or more primers) having the sequence of one of SEQ ID NOs; 29-84 and 163-209. In some embodiments, the technology described herein relates to a composition according to any of the aspects described herein in which the set of primers comprises at least one primer (e.g., one primer, two primers, three primers, four primers, five primers, six primers, or more primers) having the sequence of one of SEQ ID NOs; 29-84 and 163-209 with no more than four substitutions, deletions, and/or insertions within the entirety of the primer. In some embodiments, the technology described herein relates to a composition according to any of the aspects described herein in which the set of primers comprises at least one primer (e.g., one primer, two primers, three primers, four primers, five primers, six primers, or more primers) having the sequence of one of SEQ ID NOs; 29-84 and 163-209 with no more than four substitutions, deletions, and/or insertions within the 5′ tail region of the primer. In some embodiments, the technology described herein relates to a composition according to any of the aspects described herein in which the set of primers comprises primers having the sequence of SEQ ID NOs; 29-44, 45-56, 57-66, 67-84, 163-186, or 187-209. In some embodiments, the technology described herein relates to a composition according to any of the aspects described herein in which the set of primers comprises primers having the sequence of SEQ ID NOs; 29-44, 45-56, 57-66, 67-84, 163-186, or 187-209 with no more than four substitutions, deletions, and/or insertions within the entirety of the primer. In some embodiments, the technology described herein relates to a composition according to any of the aspects described herein in which the set of primers comprises primers having the sequence of SEQ ID NOs; 29-44, 45-56, 57-66, 67-84, 163-186, or 187-209 with no more than four substitutions, deletions, and/or insertions within the 5′ tail region of the primer. In the context of this paragraph, an insertion refers to the insertion of only one nucleotide, e.g. 2 insertions, even at the same site, refers to the addition of only 2 nucleotides. Similarly, in the context of this paragraph, a deletion refers to the deletion of only one nucleotide, e.g. 2 deletions, even at the same site, refers to the deletion of only 2 nucleotides. A primer bearing substitutions, insertions, or deletions relative to one of SEQ ID Nos: 29-44, 45-56, 57-66, 67-84, 163-186, or 187-209 must retain the ability to specifically hybridize to the complement of the 3′ core region of SEQ ID NOs: 29-44, 45-56, 57-66, 67-84, 163-186, or 187-209 under initial reaction phase annealing conditions as described herein.

Detection

In various embodiments, the methods and compositions described herein relate to detecting amplified products for each target polynucleotide, target polymorphism, and/or allelic target site locus. In some embodiments, the detecting of the amplified product for each polynucleotide affirmatively indicates the presence of the polynucleotide in a sample. In some embodiments, the detecting of an amplified product of a polymorphic target polynucleotide indicates the presence of a particular allele of that polymorphic target polynucleotide in the sample.

In some embodiments, the detecting of the amplified product for each allelic target site locus indicates the genotype at the target site locus. By way of non-limiting example, if a sample is provided comprising a single copy of a target site locus which can be of genotype A or genotype B, a PCR amplification regimen according to the methods described herein can be performed with a set of oligonucleotide primers specific to the target site locus that comprises a dual domain primer with a 3′ terminal nucleotide(s) specific for the sequence comprising genotype A, a dual domain primer with a 3′ terminal nucleic acid(s) specific for the sequence comprising genotype B, and a reverse amplifying primer. After the final cycle of a PCR amplification regimen, the amplification products are detected and 2 outcomes are possible. If the product of the dual domain primer specific for genotype A is detected, the sample is indicated to be of genotype A. If the product of the dual domain primer specific for genotype B is detected, the sample is indicated to be of genotype B. One of skill in the art will appreciate that this principle can be applied to scenarios wherein there are multiple possible alleles of a target site locus, or multiple copies of a target site locus present in a sample, (e.g. in the case of a sample from a diploid organism). Also, for instances where there are only two alleles known to exist, it is contemplated that the assay can be performed without one or the other of the dual domain primers or truncated dual domain primers specific for genotype A or genotype B. In this instance, e.g., if only primers specific for genotype A are used, detection of extension/amplification product with the genotype A-specific primer affirmatively indicates genotype A, and lack of product passively indicates genotype B.

The methods and compositions described herein relate to the amplified products of two or more members of a primer subset which should be distinguishable. In some embodiments, the methods and compositions described herein relate to PCR amplification regimens wherein the amplified products of two or more members of a primer subset can be distinguished by being of distinct sizes. As used herein, a nucleic acid is of a “distinct size” if it is resolvable from nucleic acids of a different size. “Different sizes” refers to nucleic acid molecules that differ by at least one nucleotide in length. Generally, distinctly sized amplification products useful according to the methods described herein differ by a number of nucleotides greater than or equal to the limit of resolution for the separation process used in a given separation or detection method. For example, when the limit of resolution of separation is one base, distinctly sized amplification products differ by at least one base in length, but can differ by 2 bases, 5 bases, 10 bases, 20 bases, 50 bases, 100 bases or more. When the limit of resolution is, for example, 10 bases, distinctly sized amplification products will differ by at least 10 bases, but can differ by 11 bases 15 bases, 20 bases, 30 bases, 50 bases, 100 bases or more.

The amplified products of two or more members of a primer can be designed to be of distinct sizes if, for example, the primer set included one reverse primer and two forward primers, each of the forward primers being of a different length. In some embodiments, the 3′ core region of the two or more members of a primer subset can be of different lengths. However, it is preferred that the 5′ tail region of the two or more members of a primer subset can be of different lengths. In some embodiments, the overall lengths of two or more members of a primer subset can vary.

In some embodiments, the amplified products of two or more members of a primer subset can be of distinct sizes if, for example, the primer set includes one reverse primer and two forward primers, each of the forward primers has a 3′ core region that recognizes a different nucleotide sequence of the target polynucleotide.

In some embodiments, both the lengths of the primers or any portion thereof and the lengths of the segment of the target polynucleotide sequence that they anneal to can vary. Variation in the length of target sequence amplified, e.g. by chosen placement of the forward and reverse primers further or closer apart, is a straightforward approach to ensuring ready distinctions between products from different target loci. Variation in the length of the primer, especially the 5′ tail regions of dual domain primers, is particularly effective, e.g. distinguishing the products of specific alleles of a given target locus in an assay.

In some embodiments the amplified products are distinguished by being labeled with different detectable labels. In some embodiments, the label is incorporated into a dual domain or truncated dual domain primer. In some embodiments, the label is conjugated to a dual domain or truncated dual domain primer.

In some embodiments, the label is bound to the primer after the PCR amplification regimen is complete. In some embodiments, the label is conjugated to an oligonucleotide or antibody or portion thereof that specifically binds to primer, or to a moiety attached thereto.

In some embodiments, the label is bound to the 3′ core region of a dual domain or truncated dual domain primer after the PCR amplification regimen is complete. In some embodiments, the label is conjugated to an oligonucleotide or antibody or portion thereof that specifically binds to the 3′ core region of a dual domain primer or truncated dual domain primer, or to a moiety attached thereto.

In some embodiments, the label is bound to the 5′ tail region of a dual domain or truncated dual domain primer after the PCR amplification regimen is complete. In some embodiments, the label is conjugated to an oligonucleotide or antibody or portion thereof that specifically binds to the 5′ tail region of a dual domain primer or truncated dual domain primer, or to a moiety attached thereto.

Two detectable labels are considered different if the signal from one label can be distinguished from the signal from the other. Detectable labels can comprise, for example, a light-absorbing dye, a fluorescent dye, or a radioactive label. Fluorescent dyes are preferred. Generally, a fluorescent signal is distinguishable from another fluorescent signal if the peak emission wavelengths are separated by at least 20 nm. Greater peak separation is preferred, especially where the emission peaks of fluorophores in a given reaction are wide, as opposed to narrow or more abrupt peaks.

Detectable labels, methods of detecting them, and methods of incorporating them into or coupling and/or binding them to an amplified product are well known in the art. The following is provided by way of non-limiting example.

In some embodiments, detectable labels can include labels that can be detected by spectroscopic, photochemical, biochemical, immunochemical, electromagnetic, radiochemical, or chemical means, such as fluorescence, chemifluoresence, or chemiluminescence, or any other appropriate means.

The detectable labels used in the methods described herein can be primary labels (where the label comprises a moiety that is directly detectable or that produces a directly detectable moiety) or secondary labels (where the detectable label binds to another moiety to produce a detectable signal, e.g., as is common in immunological labeling using secondary and tertiary antibodies).

The detectable label can be linked by covalent or non-covalent means to nucleic acids. Alternatively, a detectable label can be linked such as by directly labeling a molecule that achieves binding to another nucleic acid via a ligand-receptor binding pair arrangement or other such specific recognition molecules. Detectable labels can include, but are not limited to radioisotopes, bioluminescent compounds, chromophores, antibodies, chemiluminescent compounds, fluorescent compounds, metal chelates, and enzymes.

In some embodiments, a detectable label can be a fluorescent dye molecule, or fluorophore including, but not limited to fluorescein, phycoerytllrin Cy3™, Cy5™, allophycocyanine, Texas Red, peridenin chlorophyll, cyanine, tandem conjugates such as phycoerythrin-Cy5™, green fluorescent protein, rhodamine, fluorescein isothiocyanate (FITC) and Oregon Green™, rhodamine and derivatives (e.g., Texas red and tetrarhodimine isothiocynate (TRITC)), biotin, phycoerythrin, AMCA, CyDyes™, 6-carboxyfhiorescein (commonly known by the abbreviations FAM and F), 6-carboxy-2′,4′,7′,4,7-hexachlorofiuorescein (HEX), 6-carboxy-4′,5′-dichloro-2′,7′-dimethoxyfiuorescein (JOE or J), N,N,N′,N′-tetramethyl-6carboxyrhodamine (TAMRA or T), 6-carboxy-X-rhodamine (ROX or R), 5-carboxyrhodamine-6G (R6G5 or G5), 6-carboxyrhodamine-6G (R6G6 or G6), and rhodamine 110; cyanine dyes. e.g. Cy3, Cy5 and Cy7 dyes; coumarins, e.g. umbelliferone; benzimide dyes, e.g. Hoechst 33258; phenanthridine dyes, e.g. Texas Red; ethidium dyes; acridine dyes carbazole dyes; phenoxazine dyes; porphyrin dyes; polymethine dyes, e.g. cyanine dyes such as Cy3, Cy5, etc; BODIPY dyes and quinoline dyes.

In some embodiments, a detectable label can be a radiolabel including, but not limited to ³H, ¹²⁵I, ³⁵S, ¹⁴C, ³²P, and ³³P.

In some embodiments, a detectable label can be an enzyme including, but not limited to horseradish peroxidase and alkaline phosphatase. An enzymatic label can produce, for example, a chemiluminescent signal, a color signal, or a fluorescent signal.

In some embodiments, a detectable label is a chemiluminescent label, including, but not limited to luminol, luciferin or lucigenin.

In some embodiments, a detectable label can be a spectral calorimetric label including, but not limited to colloidal gold or colored glass or plastic (e.g., polystyrene, polypropylene, and latex) beads.

In some embodiments, the methods and compositions described herein relate to PCR amplification regimens wherein the amplified products of two or more members of a primer subset can be distinguished by being sequenced. Methods of sequencing nucleic acids are well known in the art and commercial sequencing services are widely available (e.g. Genscript; Piscataway, N.J.).

In some embodiments, the methods and compositions described herein relate to PCR amplification regimens wherein the amplified products of two or more members of a primer subset can be distinguished by melting-curve analysis. Methods of melting-curve analyses are well known in the art (e.g. Ririe et al. Analytical Biochemistry 1997 245:154-160; Wittwer et al. Clinical Chemistry 2003 49:853-860; and Liew et al. Clinical Chemistry 2007 50:1156-1164; which are incorporated by reference herein in their entireties).

Direct detection of size-separated amplification products is preferred. However, in some embodiments, the methods and compositions described herein relate to PCR amplification regimens wherein the amplified products of two or more members of a primer subset can be distinguished by oligonucleotide hybridization. One having ordinary skill in the art, using the sequence information of the target polymorphisms, can design probes which are fully complementary to a single polymorphism and not to other polymorphisms of a given target site locus or target nucleic acid. Hybridization conditions can be routinely optimized to minimize background signal by non-fully complementary hybridization. Hybridization probes can be designed to hybridize to the 5′ tail region, the 3′ core region, the tail-core junction, or part of the amplified product not comprised by the primer, provided that the sequence to which the probe will hybridize distinguishes it from at least one other amplified product present in the reaction.

In some embodiments, the PCR amplification regimen described herein is a multiplex and/or multimodal regimen. In some embodiments, an amplification product of a dual domain (or truncated dual domain primer) can be distinguished from the amplification products of other dual domain (or truncated dual domain primers) by at least two approaches. By way of non-limiting example, all the products of a set of oligonucleotide primers can be labeled with one common label and each unique amplification product can be distinguished from the other amplification products of the same subset of primers by being of a distinct size.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. The teachings of the disclosure provided herein can be applied to other procedures or methods as appropriate. The various embodiments described herein can be combined to provide further embodiments. Aspects of the disclosure can be modified, if necessary, to employ the compositions, functions and concepts described herein to provide yet further embodiments of the method and compositions described herein.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

This invention is further illustrated by the following examples which should not be construed as limiting.

Some embodiments of the technology described herein can be defined according to any of the following numbered paragraphs:
1. A method for determining the presence of one or more target polynucleotides, the method comprising;
   performing a PCR amplification regimen comprising cycles of strand separation, primer annealing, and primer extension on a reaction mixture comprising a nucleic acid sample and a set of oligonucleotide primers specific for each target polynucleotide;
   wherein each said set of oligonucleotide primers comprises a first subset of at least one truncated dual domain forward primer and a second subset of at least one reverse primer;
   wherein each truncated dual domain primer of a set comprises a 5′ tail region that differs from the 5′ tail region of other dual domain primers in the set and a 3′ core region complementary to a sequence on one strand of a double-stranded nucleic acid comprising said target;
   wherein for each truncated dual domain primer, the 3′ core substantially anneals to its complementary target site sequence at a first annealing temperature, and the sequence comprised by the 5′ tail and 3′ core region substantially anneals to its complement at a second annealing temperature, the second annealing temperature being higher than the first annealing temperature, such that at said second annealing temperature said 3′ core of said truncated dual domain primer cannot substantially anneal to a template molecule that does not also have the complement of the truncated dual domain primer's 5′ tail sequence;
   wherein for each truncated dual domain primer of a primer set:
      the 5′ tail sequence does not have any homology to the target sequence; and
      the 5′ tail sequence has 6 or fewer contiguous homologous bases relative to any other 5′ tail sequence of the dual domain or truncated dual domain primer set;
   wherein said PCR amplification regimen comprises first and second phases,
      the first phase comprising annealing at said first annealing temperature for a first set of cycles, and;
      the second phase comprising annealing at said second annealing temperature for a second set of cycles; and detecting an amplified product for each target polynucleotide, wherein said detecting indicates the presence of said target polynucleotide.
2. The method of paragraph 1, wherein a reverse primer comprises an amplifying primer.
3. The method of paragraph 1, wherein said target polynucleotides are selected from the group consisting of:
   polynucleotides comprising SNPs; polynucleotides comprising alleles of SNPs; protein coding DNA or RNA polynucleotides; non-protein coding DNA or RNA polynucleotides; polynucleotides comprising mutations; polynucleotides comprising deletions; and polynucleotides comprising insertions.
4. The method of paragraph 1, wherein a set of primers comprises at least one dual domain forward primer;
   wherein each dual domain primer of a set comprises a 5′ tail region that differs from the 5′ tail region of other dual domain primers in the set, a 3′ core region complementary to a sequence on one strand of a double-stranded nucleic acid comprising said target, and a terminal nucleotide complementary to one of the variant nucleotides occurring at said target site;
   wherein for each dual domain primer, the 3′ core substantially anneals to its complementary target site sequence at a first annealing temperature, and the sequence comprised by the 5′ tail and 3′ core region substantially anneals to its complement at a second annealing temperature, the second annealing temperature being higher than the first annealing temperature, such that at said second annealing temperature said 3′ core of said dual domain primer cannot substantially anneal to a template molecule that does not also have the complement of the dual domain primer's 5′ tail sequence; and
   wherein for each truncated dual domain primer of a primer set:
      the 5′ tail sequence does not have any homology to the target sequence; and
      the 5′ tail sequence has 6 or fewer contiguous homologous bases relative to any other 5′ tail sequence of the dual domain or truncated dual domain primer set.
5. The method of any of paragraphs 1-4, wherein a plurality of target polynucleotides are detected in a single reaction.
6. The method of paragraph 5, wherein the target polynucleotides comprise at least two polynucleotides selected from the group consisting of:
   polynucleotides comprising SNPs; polynucleotides comprising alleles of SNPs; protein coding DNA or RNA polynucleotides; non-protein coding DNA or RNA polynucleotides; polynucleotides comprising mutations; polynucleotides comprising deletions; and polynucleotides comprising insertions.
7. The method of paragraph 6, wherein the target polynucleotides comprise at least one polynucleotide selected from the group consisting of:
   polynucleotides comprising SNPs and polynucleotides comprising alleles of SNPs; and at least one polynucleotide selected from the group consisting of:
   protein coding DNA or RNA polynucleotides; non-protein coding DNA or RNA polynucleotides; polynucleotides comprising mutations; polynucleotides comprising deletions; and polynucleotides comprising insertions.
8. The method of any of paragraphs 4-7, wherein at least one of the target polynucleotides comprises a SNP and wherein said method genotypes the nucleotide at said SNP.
9. The method of paragraph 8, wherein dual domain or truncated dual domain primers specific for each of 2 or more alleles of a target SNP are present in the same reaction.
10. The method of paragraph 9, wherein one dual domain or truncated dual domain primer of a set is specific for the predominant allele of the target SNP.
11. The method of any of paragraphs 1-10, wherein the dual domain or truncated dual domain primers specific for each of 2 or more alleles or sequence variants of a target nucleotide are present in the same reaction.
12. The method of any of paragraphs 1-11, wherein one dual domain or truncated dual domain primer of a set is specific for the predominant allele or variant allele of the target polynucleotide.
13. The method of any of paragraphs 1-12, wherein the amplified products of two or more members of a primer subset can be distinguished.
14. The method of paragraph 13, wherein the amplified products of two or more members of a primer subset are distinguished by being of distinct sizes.
15. The method of paragraph 13, wherein the amplified products of two or more members of a primer subset are distinguished by being labeled with different detectable labels.
16. The method of paragraph 13, wherein the products of two or more members of a primer subset are distinguished by sequencing the products.
17. The method of paragraph 13, wherein the products of two or more members of a primer subset are distinguished by oligonucleotide hybridization.
18. The method of paragraph 13, wherein the products of two or more members of a primer subset are distinguished by melting-curve analysis of the amplified products.
19. The method of any of paragraphs 1-18, wherein the core sequence is 10 to 40 nucleotides in length.
20. The method of any of paragraphs 1-19, wherein the core sequence is 11 to 20 nucleotides in length.
21. The method of any of paragraphs 1-20, wherein the first phase comprises from 1 to 10 cycles.
22. The method of any of paragraphs 1-21, wherein the first phase comprises from 3 to 5 cycles.
23. The method of any of paragraphs 1-22, wherein each 5′ tail sequence of a set has fewer than 3 contiguous homologous bases relative to the other 5′ tail sequences of the set.
24. The method of any of paragraphs 1-23, wherein each 5′ tail sequence of a set has fewer than 2 contiguous homologous bases relative to the other 5′ tail sequences of the set.
25. The method of any of paragraphs 1-24, wherein the 5′ tail sequence is from 1 to 200 nucleotides in length.
26. The method of any of paragraphs 1-25, wherein the 5′ tail sequence is from 4 to 150 nucleotides in length.
27. The method of any of paragraphs 1-26, wherein the second annealing temperature is at least 4° C. higher than the first annealing temperature.
28. The method of any of paragraphs 1-27, wherein the dual domain or truncated dual domain primers present in a reaction have Tₘ values for their respective 3′ core regions that vary by no more than 8° C. from each other.
29. The method of any of paragraphs 1-28, wherein the 5′ tail sequence is selected from any of SEQ ID NOs 210-2709.
30. The method of any of paragraphs 1-29, wherein each of the 5′ tail sequences is selected from SEQ ID NOs. 210-2709.
31. The method of any of paragraphs 1-30, wherein the one or more set of primers comprises two or more sequences selected from the groups consisting of:
   SEQ ID NOs: 29-44; SEQ ID NOs: 51-56; SEQ ID NOs: 62-66; SEQ ID NOs: 67-84; SEQ ID NOs: 163-186; and SEQ ID NOs: 187-209.
32. A composition for determining the presence of one or more target polynucleotides,
comprising;
at least one set of oligonucleotide primers specific for each target polynucleotide;
wherein each said set of oligonucleotide primers comprises a first subset of at least one truncated dual domain forward primer and a second subset of at least one reverse primer;
wherein each truncated dual domain primer of a set comprises a 5′ tail region that differs from the 5′ tail region on other truncated dual domain primers in the set and a 3′ core region complementary to a sequence on one strand of a double-stranded nucleic acid comprising said target;
wherein for each truncated dual domain primer, the 3′ core substantially anneals to its complementary target site sequence at a first annealing temperature, and the sequence comprised by the 5′ tail and 3′ core region substantially anneals to its complement at a second annealing temperature, the second annealing temperature being higher than the first annealing temperature, such that at said second annealing temperature said 3′ core of said truncated dual domain primer cannot substantially anneal to a template molecule that does not also have the complement of the primer's 5′ tail sequence;
wherein for each member of said truncated dual domain primer set:
   the 5′ tail sequence does not have any homology to the target sequence; and
   the 5′ tail sequence has 6 or fewer contiguous homologous bases relative to the other 5′ tail sequences of the truncated dual domain primer set.
33. The composition of paragraph 32, further comprising a nucleic acid sample.
34. The composition of any of paragraphs 32-33, wherein at least one forward primer comprises a dual domain primer;
   wherein each dual domain primer of a set comprises a 5′ tail region that differs from the 5′ tail region on other dual domain primers in the set, a 3′ core region complementary to a sequence on one strand of a double-stranded nucleic acid comprising said target, and a terminal nucleotide complementary to one of the variant nucleotides occurring at said target site;
   wherein for each dual domain primer, the 3′ core substantially anneals to its complementary target site sequence at a first annealing temperature, and the sequence comprised by the 5′ tail and 3′ core region substantially anneals to its complement at a second annealing temperature, the second annealing temperature being higher than the first annealing temperature, such that at said second annealing temperature said 3′ core of said dual domain primer cannot substantially anneal to a template molecule that does not also have the complement of the primer's 5′ tail sequence;
   wherein for each member of said dual domain primer set:
      the 5′ tail sequence does not have any homology to the target sequence; and
      the 5′ tail sequence has 6 or fewer contiguous homologous bases relative to the other 5′ tail sequences of the dual domain primer set.
35. The composition of any of paragraphs 32-34, wherein a reverse primer comprises a dual domain primer.
36. The composition of any of paragraphs 32-34, wherein a reverse primer comprises a truncated dual domain primer.
37. The composition of any of paragraphs 32-36, wherein a reverse primer comprises an amplifying primer.
38. The composition of any of paragraphs 32-37, wherein said target polynucleotides are selected from the group consisting of:
   polynucleotides comprising SNPs; polynucleotides comprising alleles of SNPs; protein coding DNA or RNA polynucleotides; non-protein coding DNA or RNA polynucleotides; polynucleotides comprising mutations; polynucleotides comprising deletions; and polynucleotides comprising insertions.
39. The composition of any of paragraphs 32-38, wherein the target polynucleotides comprise at least two polynucleotides selected from the group consisting of:
   polynucleotides comprising SNPs; polynucleotides comprising alleles of SNPs; protein coding DNA or RNA polynucleotides; non-protein coding DNA or RNA polynucleotides; polynucleotides comprising mutations; polynucleotides comprising deletions; and polynucleotides comprising insertions.
40. The composition of any of paragraphs 32-39, wherein the target polynucleotides comprise at least one polynucleotide selected from the group consisting of:
   polynucleotides comprising SNPs and polynucleotides comprising alleles of SNPs; and at least one polynucleotide selected from the group consisting of:
   protein coding DNA or RNA polynucleotides; non-protein coding DNA or RNA polynucleotides; polynucleotides comprising mutations; polynucleotides comprising deletions; and polynucleotides comprising insertions.
41. The composition of any of paragraphs 32-40, wherein a subset of dual domain forward primers comprises primers specific for each of 2 or more alleles of a target SNP.
42. The composition of any of paragraphs 32-41, wherein one dual domain forward primer of a set is specific for the predominant allele of the target SNP.
43. The composition of any of paragraphs 32-42, wherein the subset of dual domain or truncated dual domain forward primers comprises primers specific for each of 2 or more alleles or sequence variants of a target nucleotide.
44. The composition of any of paragraphs 32-43, wherein one dual domain or truncated dual domain forward primer of a set is specific for the predominant allele or variant allele of the target polynucleotide.
45. The composition of any of paragraphs 32-44, wherein the core sequence is 10 to 40 nucleotides in length.
46. The composition of any of paragraphs 32-45, wherein the core sequence is 11 to 20 nucleotides in length.
47. The composition of any of paragraphs 32-46, wherein each 5′ tail sequence of a set has fewer than 3 contiguous homologous bases relative to the other 5′ tail sequences of the set.
48. The composition of any of paragraphs 32-47, wherein each 5′ tail sequence of a set has fewer than 2 contiguous homologous bases relative to the other 5′ tail sequences of the set.
49. The composition of any of paragraphs 32-48, wherein the 5′ tail sequence is from 1 to 200 nucleotides in length.
50. The composition of any of paragraphs 32-49, wherein the 5′ tail sequence is from 4 to 150 nucleotides in length.
51. The composition of any of paragraphs 32-50, wherein the second annealing temperature is at least 4° C. higher than the first annealing temperature.
52. The composition of any of paragraphs 32-51, wherein the dual domain primers present in a reaction have Tₘ values for their respective 3′ core regions that vary by no more than 8° C. from each other.
53. The composition of any of paragraphs 32-52, wherein the 5′ tail sequence is selected from any of SEQ ID NOs 210-2709.
54. The compositions of any of paragraphs 32-53, wherein each of the 5′ tail sequences is selected from SEQ ID NOs. 210-2709.
55. The composition of any of paragraphs 32-54, wherein the one or more set of primers comprises two or more sequences selected from the groups consisting of:
   SEQ ID NOs: 29-44; SEQ ID NOs: 51-56; SEQ ID NOs: 62-66; SEQ ID NOs: 67-84; SEQ ID NOs: 163-186; and SEQ ID NOs: 187-209.
56. A method for determining the presence of one or more target polynucleotides, the method comprising;
   performing a PCR amplification regimen comprising cycles of strand separation, primer annealing, and primer extension on a reaction mixture comprising a nucleic acid sample and a set of oligonucleotide primers specific for each target polynucleotide;
   wherein each said set of oligonucleotide primers comprises a first subset of at least one truncated dual domain forward primer and a second subset of at least one reverse primer;
   wherein each truncated dual domain primer of a set comprises a 5′ tail region that differs from the 5′ tail region of other dual domain primers in the set and a 3′ core region complementary to a sequence on one strand of a double-stranded nucleic acid comprising said target;
   wherein for each truncated dual domain primer, the 3′ core substantially anneals to its complementary target site sequence at a first annealing temperature, and the sequence comprised by the 5′ tail and 3′ core region substantially anneals to its complement at a second annealing temperature, the second annealing temperature being higher than the first annealing temperature, such that at said second annealing temperature said 3′ core of said truncated dual domain primer cannot substantially anneal to a template molecule that does not also have the complement of the truncated dual domain primer's 5′ tail sequence;
   wherein for each truncated dual domain primer of a primer set:
      the 5′ tail sequence does not have any homology to the target sequence; and
      the 5′ tail sequence has 6 or fewer contiguous homologous bases relative to any other 5′ tail sequence of the dual domain or truncated dual domain primer set;
   wherein said PCR amplification regimen comprises first and second phases,
      the first phase comprising annealing at said first annealing temperature for a first set of cycles, and;
      the second phase comprising annealing at said second annealing temperature for a second set of cycles;
   and detecting an amplified product for each target polynucleotide, wherein said detecting indicates the presence of said target polynucleotide.
57. The method of paragraph 56, wherein a reverse primer comprises an amplifying primer.
58. The method of any of paragraphs 56-57, wherein said target polynucleotides are selected from the group consisting of:
   polynucleotides comprising SNPs; polynucleotides comprising alleles of SNPs; protein coding DNA or RNA polynucleotides; non-protein coding DNA or RNA polynucleotides; polynucleotides comprising mutations; polynucleotides comprising deletions; and polynucleotides comprising insertions.
59. The method of any of paragraphs 56-58, wherein a set of primers comprises at least one dual domain forward primer;
   wherein each dual domain primer of a set comprises a 5′ tail region that differs from the 5′ tail region of other dual domain primers in the set, a 3′ core region complementary to a sequence on one strand of a double-stranded nucleic acid comprising said target, and a terminal nucleotide complementary to one of the variant nucleotides occurring at said target site;
   wherein for each dual domain primer, the 3′ core substantially anneals to its complementary target site sequence at a first annealing temperature, and the sequence comprised by the 5′ tail and 3′ core region substantially anneals to its complement at a second annealing temperature, the second annealing temperature being higher than the first annealing temperature, such that at said second annealing temperature said 3′ core of said dual domain primer cannot substantially anneal to a template molecule that does not also have the complement of the dual domain primer's 5′ tail sequence; and
   wherein for each truncated dual domain primer of a primer set:
      the 5′ tail sequence does not have any homology to the target sequence; and
      the 5′ tail sequence has 6 or fewer contiguous homologous bases relative to any other 5′ tail sequence of the dual domain or truncated dual domain primer set.
60. The method of any of paragraphs 56-59, wherein a plurality of target polynucleotides are detected in a single reaction.
61. The method of paragraph 60, wherein the target polynucleotides comprise at least two polynucleotides selected from the group consisting of:
   polynucleotides comprising SNPs; polynucleotides comprising alleles of SNPs; protein coding DNA or RNA polynucleotides; non-protein coding DNA or RNA polynucleotides; polynucleotides comprising mutations; polynucleotides comprising deletions; and polynucleotides comprising insertions.
62. The method of paragraph 61, wherein the target polynucleotides comprise at least one polynucleotide selected from the group consisting of:
   polynucleotides comprising SNPs and polynucleotides comprising alleles of SNPs; and at least one polynucleotide selected from the group consisting of:
   protein coding DNA or RNA polynucleotides; non-protein coding DNA or RNA polynucleotides; polynucleotides comprising mutations; polynucleotides comprising deletions; and polynucleotides comprising insertions.
63. The method of any of paragraphs 56-62, wherein at least one of the target polynucleotides comprises a SNP and wherein said method genotypes the nucleotide at said SNP.
64. The method of paragraph 63, wherein dual domain or truncated dual domain primers specific for each of 2 or more alleles of a target SNP are present in the same reaction.
65. The method of paragraph 64, wherein one dual domain or truncated dual domain primer of a set is specific for the predominant allele of the target SNP.
66. The method of any of paragraphs 56-65, wherein the dual domain or truncated dual domain primers specific for each of 2 or more alleles or sequence variants of a target nucleotide are present in the same reaction.
67. The method of any of paragraphs 56-66, wherein one dual domain or truncated dual domain primer of a set is specific for the predominant allele or variant allele of the target polynucleotide.
68. The method of any of paragraphs 56-67, wherein the amplified products of two or more members of a primer subset can be distinguished.
69. The method of paragraph 68, wherein the amplified products of two or more members of a primer subset are distinguished by being of distinct sizes.
70. The method of paragraph 68, wherein the amplified products of two or more members of a primer subset are distinguished by being labeled with different detectable labels.
71. The method of paragraph 68, wherein the products of two or more members of a primer subset are distinguished by sequencing the products.
72. The method of paragraph 68, wherein the products of two or more members of a primer subset are distinguished by oligonucleotide hybridization.
73. The method of paragraph 68, wherein the products of two or more members of a primer subset are distinguished by melting-curve analysis of the amplified products.
74. The method of any of paragraphs 56-73, wherein the core sequence is 10 to 40 nucleotides in length.
75. The method of any of paragraphs 56-74, wherein the core sequence is 11 to 20 nucleotides in length.
76. The method of any of paragraphs 56-75, wherein the first phase comprises from 1 to 10 cycles.
77. The method of any of paragraphs 56-76, wherein the first phase comprises from 3 to 5 cycles.
78. The method of any of paragraphs 56-77, wherein each 5′ tail sequence of a set has fewer than 3 contiguous homologous bases relative to the other 5′ tail sequences of the set.
79. The method of any of paragraphs 56-78, wherein each 5′ tail sequence of a set has fewer than 2 contiguous homologous bases relative to the other 5′ tail sequences of the set.
80. The method of any of paragraphs 56-79, wherein the 5′ tail sequence is from 1 to 200 nucleotides in length.
81. The method of any of paragraphs 56-80, wherein the 5′ tail sequence is from 4 to 150 nucleotides in length.
82. The method of any of paragraphs 56-81, wherein the second annealing temperature is at least 4° C. higher than the first annealing temperature.
83. The method of any of paragraphs 56-82, wherein the dual domain or truncated dual domain primers present in a reaction have Tₘ values for their respective 3′ core regions that vary by no more than 8° C. from each other.
84. The method of any of paragraphs 56-83, wherein the 5′ tail sequence is selected from any of SEQ ID NOs 210-2709.
85. The method of any of paragraphs 56-84, wherein each of the 5′ tail sequences is selected from SEQ ID NOs. 210-2709.
86. The method of any of paragraphs 56-85, wherein the one or more set of primers comprises two or more sequences selected from the groups consisting of:
   SEQ ID NOs: 29-44; SEQ ID NOs: 51-56; SEQ ID NOs: 62-66; SEQ ID NOs: 67-84; SEQ ID NOs: 163-186; and SEQ ID NOs: 187-209.
87. A composition for determining the presence of one or more target polynucleotides,
   comprising;
   at least one set of oligonucleotide primers specific for each target polynucleotide;
   wherein each said set of oligonucleotide primers comprises a first subset of at least one truncated dual domain forward primer and a second subset of at least one reverse primer;
   wherein each truncated dual domain primer of a set comprises a 5′ tail region that differs from the 5′ tail region on other truncated dual domain primers in the set and a 3′ core region complementary to a sequence on one strand of a double-stranded nucleic acid comprising said target;
   wherein for each truncated dual domain primer, the 3′ core substantially anneals to its complementary target site sequence at a first annealing temperature, and the sequence comprised by the 5′ tail and 3′ core region substantially anneals to its complement at a second annealing temperature, the second annealing temperature being higher than the first annealing temperature, such that at said second annealing temperature said 3′ core of said truncated dual domain primer cannot substantially anneal to a template molecule that does not also have the complement of the primer's 5′ tail sequence;
   wherein for each member of said truncated dual domain primer set:
      the 5′ tail sequence does not have any homology to the target sequence; and
      the 5′ tail sequence has 6 or fewer contiguous homologous bases relative to the other 5′ tail sequences of the truncated dual domain primer set.
88. The composition of paragraph 87, further comprising a nucleic acid sample.
89. The composition of any of paragraphs 87-88, wherein at least one forward primer comprises a dual domain primer;
   wherein each dual domain primer of a set comprises a 5′ tail region that differs from the 5′ tail region on other dual domain primers in the set, a 3′ core region complementary to a sequence on one strand of a double-stranded nucleic acid comprising said target, and a terminal nucleotide complementary to one of the variant nucleotides occurring at said target site;
   wherein for each dual domain primer, the 3′ core substantially anneals to its complementary target site sequence at a first annealing temperature, and the sequence comprised by the 5′ tail and 3′ core region substantially anneals to its complement at a second annealing temperature, the second annealing temperature being higher than the first annealing temperature, such that at said second annealing temperature said 3′ core of said dual domain primer cannot substantially anneal to a template molecule that does not also have the complement of the primer's 5′ tail sequence;
   wherein for each member of said dual domain primer set:
      the 5′ tail sequence does not have any homology to the target sequence; and
      the 5′ tail sequence has 6 or fewer contiguous homologous bases relative to the other 5′ tail sequences of the dual domain primer set.
90. The composition of any of paragraphs 87-89, wherein a reverse primer comprises a dual domain primer.
91. The composition of any of paragraphs 87-90, wherein a reverse primer comprises a truncated dual domain primer.
92. The composition of any of paragraphs 87-91, wherein a reverse primer comprises an amplifying primer.
93. The composition of any of paragraphs 87-92, wherein said target polynucleotides are selected from the group consisting of:
   polynucleotides comprising SNPs; polynucleotides comprising alleles of SNPs; protein coding DNA or RNA polynucleotides; non-protein coding DNA or RNA polynucleotides; polynucleotides comprising mutations; polynucleotides comprising deletions; and polynucleotides comprising insertions.
94. The composition of any of paragraphs 87-93, wherein the target polynucleotides comprise at least two polynucleotides selected from the group consisting of:
   polynucleotides comprising SNPs; polynucleotides comprising alleles of SNPs; protein coding DNA or RNA polynucleotides; non-protein coding DNA or RNA polynucleotides; polynucleotides comprising mutations; polynucleotides comprising deletions; and polynucleotides comprising insertions.
95. The composition of any of paragraphs 87-94, wherein the target polynucleotides comprise at least one polynucleotide selected from the group consisting of:
   polynucleotides comprising SNPs and polynucleotides comprising alleles of SNPs; and at least one polynucleotide selected from the group consisting of:
   protein coding DNA or RNA polynucleotides; non-protein coding DNA or RNA polynucleotides; polynucleotides comprising mutations; polynucleotides comprising deletions; and polynucleotides comprising insertions.
96. The composition of any of paragraphs 87-95, wherein a subset of dual domain forward primers comprises primers specific for each of 2 or more alleles of a target SNP.
97. The composition of any of paragraphs 87-96, wherein one dual domain forward primer of a set is specific for the predominant allele of the target SNP.
98. The composition of any of paragraphs 87-97, wherein the subset of dual domain or truncated dual domain forward primers comprises primers specific for each of 2 or more alleles or sequence variants of a target nucleotide.
99. The composition of any of paragraphs 87-98, wherein one dual domain or truncated dual domain forward primer of a set is specific for the predominant allele or variant allele of the target polynucleotide.
100. The composition of any of paragraphs 87-99, wherein the core sequence is 10 to 40 nucleotides in length.
101. The composition of any of paragraphs 87-100, wherein the core sequence is 11 to 20 nucleotides in length.
102. The composition of any of paragraphs 87-101, wherein each 5′ tail sequence of a set has fewer than 3 contiguous homologous bases relative to the other 5′ tail sequences of the set.
103. The composition of any of paragraphs 87-102, wherein each 5′ tail sequence of a set has fewer than 2 contiguous homologous bases relative to the other 5′ tail sequences of the set.
104. The composition of any of paragraphs 87-103, wherein the 5′ tail sequence is from 1 to 200 nucleotides in length.
105. The composition of any of paragraphs 87-104, wherein the 5′ tail sequence is from 4 to 150 nucleotides in length.
106. The composition of any of paragraphs 87-105, wherein the second annealing temperature is at least 4° C. higher than the first annealing temperature.
107. The composition of any of paragraphs 87-106, wherein the dual domain primers present in a reaction have Tₘ values for their respective 3′ core regions that vary by no more than 8° C. from each other.
108. The composition of any of paragraphs 87-107, wherein the 5′ tail sequence is selected from any of SEQ ID NOs 210-2709.
109. The composition of any of paragraphs 87-108, wherein each of the 5′ tail sequences is selected from SEQ ID NOs. 210-2709.
110. The composition of any of paragraphs 87-109, wherein the one or more set of primers comprises two or more sequences selected from the groups consisting of:
   SEQ ID NOs: 29-44; SEQ ID NOs: 51-56; SEQ ID NOs: 62-66; SEQ ID NOs: 67-84; SEQ ID NOs: 163-186; and SEQ ID NOs: 187-209.

### EXAMPLES

### Example 1

### KRAS/BRAF Multiplex Assay

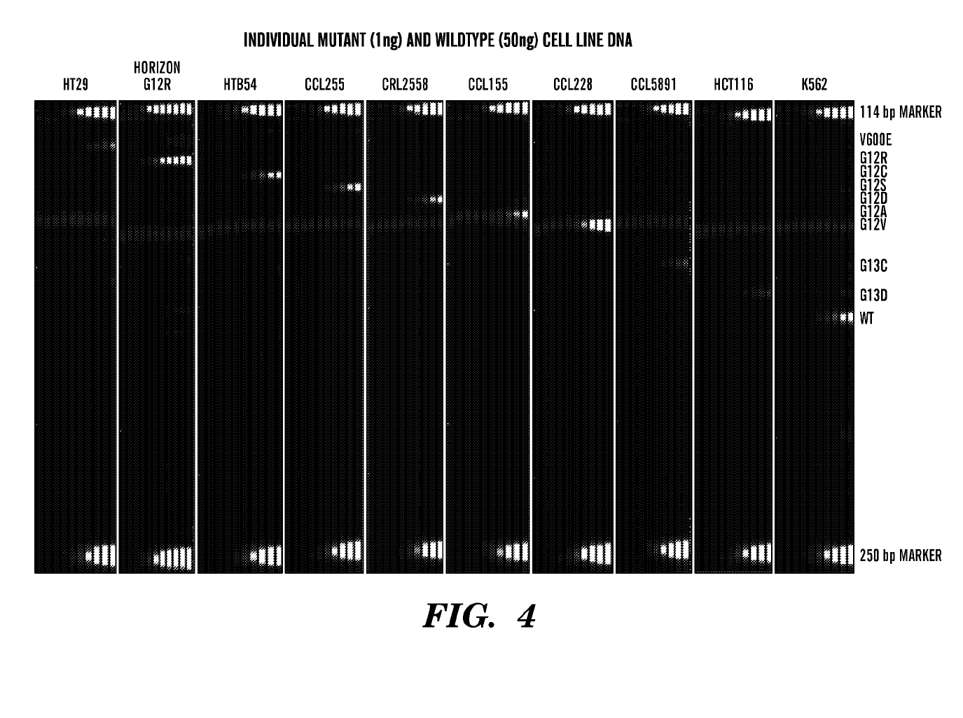

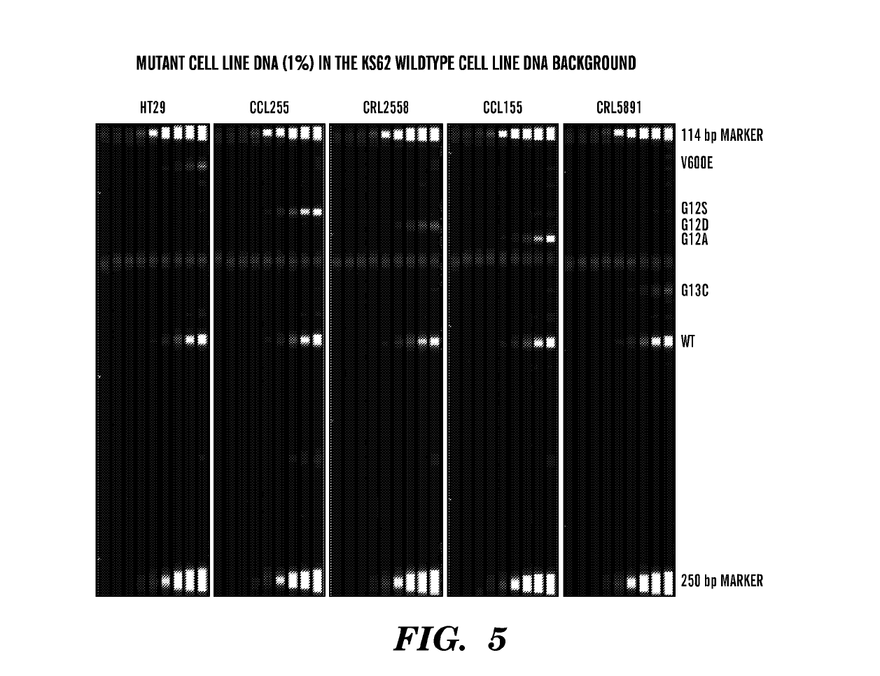

A multiplex assay was performed using dual domain primers specific for V600E/D alleles of the BRAF loci and numerous alleles of KRAS comprising wild-type and mutant sequences in the 12^{th} and 13^{th} codon of KRAS. The primer sequences are shown in Table 2. Conditions for the two phase PCR amplification regimen are shown in Tables 3, 4, and 5. The results are depicted in FIGS. 4 and 5. FIG. 4 demonstrates that sets of primers as described herein do not amplify off-target sequences and specifically amplify target alleles when the targets are present. FIG. 5 demonstrates that when multiple targets are present, a multiplexed PCR reaction performed according to the methods described herein will specifically detect the presence of target alleles without any detectable background. Both FIGS. 4 and 5 also demonstrate that no “cross-talk” amplification occurs to a detectable level when one or more than one amplified target are accumulated to a high level.

### Example 2

### IL-10/TNFα Mulitplex Assay

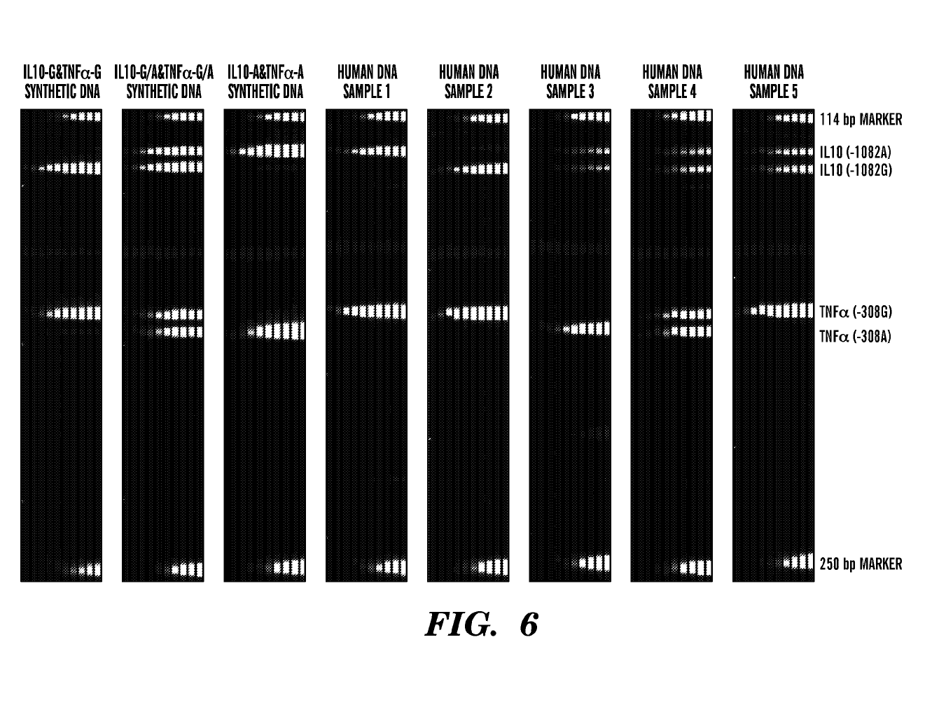

A multiplex assay was performed using dual domain primers specific for either the wild-type or −1028A and −1028G alleles of the IL-10 locus and the wild-type and −308G and −308A alleles of the TNFα locus. The primer sequences are shown in Table 6. Conditions for the two phase PCR amplification regimen are shown in Tables 7, 8, and 9. The results are depicted in FIG. 6. FIG. 6 demonstrates that sets of primers used in a multiplex reaction as described herein do not amplify off-target sequences and specifically amplify target alleles when the targets are present without detectable background amplification. FIG. 6 also demonstrates that no “cross-talk” amplification occurs to a detectable level when one or more than one amplified target are accumulated to a high level.

### Example 3

### Multimodal Assay

A multimodal assay can be performed to detect presence of the −308G and −308A alleles of the TNFα locus and the expression of IL-10 in the same reaction. First, the sample is subjected to a reverse transcriptase PCR(RT-PCR) regimen using an oligo dT primer. Briefly, a sample is mixed with reverse transcriptase, dNTPs, buffer and an oligo-dT primer. The sample is incubated at a temperature appropriate for the particular RT enzyme being used (e.g. 42° C.) and cDNA is synthesized. A commercial kit for RT PCR can be used, for example the SUPERSCRIPT™ cDNA Synthesis Kit (Cat No 11754-050; Invitrogen; Grand Island, N.Y.).

A PCR amplification regimen can then be performed. The primer sequences are shown in Table 10. The IL-10 primers span exon-intron junctions and will therefore not amplify DNA present in the same sample, permitting the specific detection of IL-10 mRNA. Conditions for the two phase PCR amplification regimen are shown in Tables 11 and 12. Production of amplified products can be detected by capillary electrophoresis separation with sizes of products distinguishable; TNF-α alleles (173 bp product=308G allele; 178 bp product=308A allele) and the presence and amount of IL-10 mRNA evidenced by the 101 bp product. Note that in any of the approaches described above, quantitation can be derived from the amplification curve generated by repeated sampling of the amplification reaction during the PCR cycling.

### Example 4

### Plasma Sample Assay

Plasma or serum specimens have been explored for diagnostics of various diseases or disorders. Diagnostics based on plasma and/or serum samples are particularly beneficial since such methods are significantly less invasive than those using biopsy tissues, such as tumor samples in cancer tests. A blood serum or plasma assay that identified tumor nucleic acids has the additional benefit of potentially detecting tumors at an earlier stage on the basis of circulating nucleic acids alone. However, plasma presents difficulties because it has a high background level of nucleic acids, and tumor cell nucleic acids will be very rare. Therefore, assays for plasma samples must be sensitive enough to detect tumor markers amongst a background of excess non-related nucleic acids. Demonstrated in this Example is that the methods and compositions described herein can successfully detect a desired target, including identification of a SNP genotype, in a plasma sample.

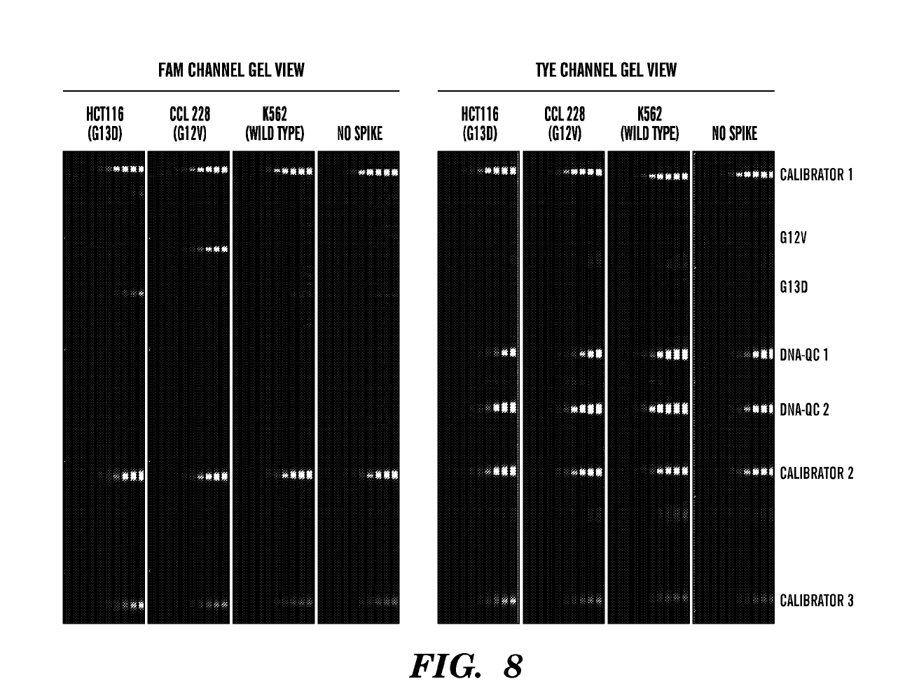

Donor plasma samples (1 mL each) were spiked with (a) 10 ng mutant DNA (CCL228 for KRAS G12V or HCT116 for KRAS G13D) (b) 200 ng K562 wild-type DNA and (c) water as a no-spike control. Samples were extracted with QIAAMP CIRCULATING NUCLEIC ACID KIT™ from Qiagen per manufacturer's instruction. No carrier RNA was used in the purification. Nucleic acid was eluted in 35 μL. 10 μL was used for PCR (50 μL/reaction). Appropriate primers are shown in Table 13 and thermocycling conditions are shown in Table 14. The detection of the target SNPs is demonstrated in FIG. 8.

Tables 15 and 16 contain additional sets of primers suitable for the detection of KRAS and BRAF SNPs in multimodal assays as described above herein, e.g. in Examples 1 and 4.

TABLE 1
Advantage of Using Short Primers
(KRAS mutant-specific primers as examples)

| | | Differentiation | |
|---|---|---|---|
| Kras Sequence around Codon 12 | Tm † | Power (DP) * | SEQ ID NO: |
| GTTGGATCATATTCGTCCACAAAATGATTCTGAATT | 69.6° C. | N/A (Wildtype) | 1 |
| AGCTGTATCGTCAAGGCACTCTTGCCTACGCCACC | | | |
| GTTGGATCATATTCGTCCACAAAATGATTCTGAATT | 69.4° C. | Δ° C. = 0.2 | 2 |
| AGCTGTATCGTCAAGGCACTCTTGCCTACGCCACT | | | |
| GTTGGATCATATTCGTCCACAAAATGATTCTGAATT | 69.3° C. | Δ° C. = 0.3 | 3 |
| AGCTGTATCGTCAAGGCACTCTTGCCTACGCCACG | | | |
| GTTGGATCATATTCGTCCACAAAATGATTCTGAATT | 69.4° C. | Δ° C. = 0.2 | 4 |
| AGCTGTATCGTCAAGGCACTCTTGCCTACGCCACA | | | |
| ATCGTCAAGGCACTCTTGCCTACGCCACC | 66.8° C. | N/A (Wildtype) | 5 |
| ATCGTCAAGGCACTCTTGCCTACGCCACT | 66.0° C. | Δ° C. = 0.8 | 6 |
| ATCGTCAAGGCACTCTTGCCTACGCCACG | 65.6° C. | Δ° C. = 1.2 | 7 |
| ATCGTCAAGGCACTCTTGCCTACGCCACA | 66.0° C. | Δ° C. = 0.8 | 8 |
| GCCTACGCCACC | 48.7° C. | N/A (Wildtype) | 9 |
| GCCTACGCCACT | 44.7° C. | Δ° C. = 4.0 | 10 |
| GCCTACGCCACG | 44.4° C. | Δ° C. = 4.3 | 11 |
| GCCTACGCCACA | 45.1° C. | Δ° C. = 3.6 | 12 |

† Tm determined with the OligoAnalyzer 3.1/TM Mismatch program from the IDT website (www.idtdna.com) using the wildtype sequence with C changed to T, G, or A at the 3′ end
* DP = Tm of Mutant primer − Tm of Wildtype primer; the larger the values, the higher difference between the two Tm's, and thus the higher differentiation power of the mutant primers against the wild-type sequence

TABLE 2
Primer sequences and amplicon sizes for detection of single-nucleotide point
mutations in codons 12 & 13 of the KRAS and codon 600 of the BRAF genes.

| | | SEQ | | SEQ | |
|---|---|---|---|---|---|
| | Forward | ID | | ID | Size |
| Mutation | Primer | NO: | Reverse primer | NO: | (bp) |
| Braf | CCGCATTT | 13 | CCTATCGAATCAGTGGAAAAATAGC | 29 | 120 |
| V600E/D | TGGTCTAG | | | | |
| | CTACAG [symbol] | | | | |
| Wild- | GGTGATTT | 14 | AAAATTTAATCAGTGGAAAAATAGC | 30 | |
| type | TGGTCTAG | | | | |
| sequence | CTACAG [symbol] | | | | |
| (for | | | | | |
| compar- | | | | | |
| ison) | | | | | |
| Kras | CGCCTAGT | 15 | TCGCTGCCTACGCCAC [symbol] | 31 | 130 |
| G12R | GTATTAAC | | | | |
| | CTTATGTG | | | | |
| | TG | | | | |
| Kras | CGCCTAGT | 16 | CATTTGTTTGCCTACGCCAC [symbol] | 32 | 134 |
| G12C | GTATTAAC | | | | |
| | CTTATGTG | | | | |
| | TG | | | | |
| Kras | CGCCTAGT | 17 | CATTATACGAGTTGCCTACGCCAC [symbol] | 33 | 138 |
| G12S | GTATTAAC | | | | |
| | CTTATGTG | | | | |
| | TG | | | | |
| Kras | CGCCTAGT | 18 | CAATATCTAATGTCCTTGCCTACGCCA [symbol] | 34 | 142 |
| G12D | GTATTAAC | | | | |
| | CTTATGTG | | | | |
| | TG | | | | |
| Kras | CGCCTAGT | 19 | ACTATCGACGTCTTGCAACTTGCCTACGCCA [symbol] | 35 | 146 |
| G12A | GTATTAAC | | | | |
| | CTTATGTG | | | | |
| | TG | | | | |
| Kras | CGCCTAGT | 20 | CATATAACGGTCGAGGACTCTACTTGCCTACGCCA [symbol] | 36 | 150 |
| G12V | GTATTAAC | | | | |
| | CTTATGTG | | | | |
| | TG | | | | |
| Kras | CGCCTAGT | 21 | CTTCGACTTCAACGAAGTTGATGTCTCTTGCCTACGC [symbol] | 37 | 154 |
| G13R | GTATTAAC | | | | |
| | CTTATGTG | | | | |
| | TG | | | | |
| Kras | CGCCTAGT | 22 | CAATAATTAATTAACGTATAAATTCCCACTCTTGCCTACGC [symbol] | 38 | 158 |
| G13S | GTATTAAC | | | | |
| | CTTATGTG | | | | |
| | TG | | | | |
| Kras | CGCCTAGT | 23 | CTGTGACAACTTCATTGATCTGTTAAGTATTACTCTTGCCTACGC [symbol] | 39 | 162 |
| G13C | GTATTAAC | | | | |
| | CTTATGTG | | | | |
| | TG | | | | |
| Kras | CGCCTAGT | 24 | CATCTCACTCATCATTACTAACTAGATAAACTTTCACTCTTGCCTACG [symbol] | 40 | 166 |
| G13A | GTATTAAC | | | | |
| | CTTATGTG | | | | |
| | TG | | | | |
| Kras | CGCCTAGT | 25 | CAAATTCTTTCATAAAATTCAATCTAAGTCATCTTTAGCACTCTTGCCTACG [symbol] | 41 | 170 |
| G13D | GTATTAAC | | | | |
| | CTTATGTG | | | | |
| | TG | | | | |
| Kras | CGCCTAGT | 26 | CAATACTTTAGCTTGTCGATACTCAGTACAAATAGTGACTAGCACTCTTGCCTACG [symbol] | 42 | 174 |
| G13V | GTATTAAC | | | | |
| | CTTATGTG | | | | |
| | TG | | | | |
| Kras | CGCCTAGT | 27 | CATATCAATGAGCATGTGAAGCATGACTCCTTTATACCTAGATCATTTAGAATGCCTACG [symbol] A [symbol] | 43 | 178 |
| WT | GTATTAAC | | | | |
| | CTTATGTG | | | | |
| | TG | | | | |
| Wild- | TTGATAGT | 28 | TCATATTCGTCCACAAAATGATTCTGAATTAGCTGTATCGTCAAGGCACTCTTGCCTACGCCACC | 44 | |
| type | GTATTAAC | | | | |
| sequence | CTTATGTG | | | | |
| (for | TG | | | | |
| compar- | | | | | |
| ison) | | | | | |

Note:
Unbold/Underlined: Tail sequence (5′ to 3′);
Bold: Target sequence (5′ to 3′);
Bold/Italicized/Underlined: Mutation site.

TABLE 3
Concentrations of primers used in the
Multiplex KRAS/BRAF Mutation Assay.

| | Final Concen- |
|---|---|
| Primer | tration (μM) |
| Braf V600E | 0.235 |
| Kras G12R | 0.220 |
| Kras G12C | 0.280 |
| Kras G12S | 0.225 |
| Kras G12D | 0.295 |
| Kras G12A | 0.220 |
| Kras G12V | 0.220 |
| Kras G13R | 0.200 |
| Kras G13S | 0.195 |
| Kras G13C | 0.145 |
| Kras G13A | 0.150 |
| Kras G13D | 0.195 |
| Kras G13V | 0.200 |
| Kras G12/13-WT | 0.067 |
| Kras12/13F | 0.350 |
| Braf600R | 0.400 |

TABLE 4
Reagent recipe for Multiplex KRAS/BRAF Mutation Assay.

| Reagent | Final concentration |
|---|---|
| FASTSTART ™ Buffer (Without Mg) | 1X |
| (Hoffmann-La Roche; Basel, Switzerland) | |
| MgCl₂ | 1.7 mM |
| dNTPs | 0.2 mM each |
| Kras/Braf Primer Mix | 0.067 to 0.400 μM |
| PrimeraDx Universal Calibrators | 0.35X |
| FASTSTART ™ Taq (Hoffmann- | 0.9 U |
| La Roche; Basel, Switzerland) | |
| DNA/water | To a final volume of 25 μl |

TABLE 5
Thermocycling profile for Multiplex
KRAS/BRAF Mutation Assay.

| | Temperature | Duration | Number |
|---|---|---|---|
| Step | (° C.) | (sec) | of Cycles |
| Pre-Heating | 96 | 360 | 1 |
| Amplification Phase 1 | 47/68/98 | 10/10/3 | 4 |
| | (anneal/extend/ | | |
| | strand separation) | | |
| Amplification Phase 2 | 53/72/98 | 5/5/3 | 17 |
| | (anneal/extend/ | | |
| | strand separation) | | |
| Amplification with | 53/72/98 | 5/220/3 | 19 |
| Sample Injection (for | (anneal/extend/ | | |
| Capillary Electropho- | strand separation) | | |
| resis Separation) | | | |

TABLE 6
Multiplex primer sequences and amplicon
sizes for detection of single-nucleotide
polymorphisms of the IL-10 and TNF-α genes.

| | | SEQ ID | | SEQ ID | Size |
|---|---|---|---|---|---|
| Mutation | Forward Primer | NO: | Reverse Primer | NO: | (bp) |
| IL-10 (-1082A) | CGAGGCTTCTTTGGGAA | 45 | GGAAGATGGGGTGGA | 51 | 123 |
| | | | AGA | | |
| IL-10 (-1082G) | TAATTATAGGCTTCTTTGG | 46 | GGAAGATGGGGTGGA | 52 | 128 |
| | GA [symbol] | | AGA | | |
| Wild-type | ACTACTAAGGCTTCTTTGG | 47 | AAAAGATGGGGTGGA | 53 | |
| sequence (for | GA( [symbol] / [symbol] ) | | AGA | | |
| comparison) | | | | | |
| TNF-α (-308G) | AGCCAACCCCGTTTTCT | 48 | CGTGAACCCCGTCC [symbol] | 54 | 173 |
| TNF-α (-308A) | AGCCAACCCCGTTTTCT | 49 | ATTTAACTGAACCCC | 55 | 178 |
| | | | GTCC [symbol] | | |
| Wild-type | CTCCAACCCCGTTTTCT | 50 | TGGAGGCTGAACCCC | 56 | |
| sequence (for | | | GTCC( [symbol] / [symbol] ) | | |
| comparison) | | | | | |

TABLE 7
Concentrations of primers used in Multiplex IL-10
(−1082)/TNF-α (−308) SNP Assay.

| | Final Concen- |
|---|---|
| Primer | tration (μM) |
| IL10-G | 0.45 |
| IL10-A | 0.28 |
| IL10-R | 0.35 |
| TNFα-G | 0.20 |
| TNFα-A | 0.35 |
| TNFα-F | 0.35 |

TABLE 8
Reagent recipe for Multiplex IL-10 (−1082)/TNF-
α (−308) SNP Assay.

| Reagent | Final concentration |
|---|---|
| FASTSTART ™ Buffer (Without Mg) | 1X |
| (Hoffmann-La Roche; Basel, Switzerland) | |
| MgCl₂ | 1.7 mM |
| dNTPs | 0.2 mM each |
| Kras/Braf Primer Mix | 0.20 to 0.45 μM |
| PrimeraDx Universal Calibrators | 0.35X |
| FASTSTART ™ Taq (Hoffmann- | 0.9 U |
| La Roche; Basel, Switzerland) | |
| DNA/water | To a final volume of 25 μl |

TABLE 9
Thermocycling profile for Multiplex IL-10 (−1082)/TNF-
α (−308) SNP Assay.

| | Temperature | Duration | Number |
|---|---|---|---|
| Step | (° C.) | (sec) | of Cycles |
| Pre-heating | 96 | 360 | 1 |
| Amplification Phase 1 | 46/68/98 | 10/10/3 | 4 |
| | (anneal/extend/ | | |
| | strand separation) | | |
| Amplification Phase 2 | 54/72/98 | 5/5/3 | 17 |
| | (anneal/extend/ | | |
| | strand separation) | | |
| Amplification with | 54/72/98 | 5/220/3 | 19 |
| Sample Injection (for | (anneal/extend/ | | |
| Capillary Electropho- | strand separation) | | |
| resis Separation) | | | |

Table 10
Multimodal primer sequences and amplicon sizes

| | | SEQ | | SEQ | |
|---|---|---|---|---|---|
| | Forward | ID | Reverse | ID | Size |
| Target | Primer | NO | primer | NO | (bP) |
| IL-10 | TCAAAGC | 57 | ACACCAT | 62 | 88 bp |
| | GGCGCTG | | CTTTCTC | | amplicon |
| | TCATC | | TTGGAGC | | 101 bp |
| | | | TTA | | Product |
| | | | | | including |
| | | | | | tail |
| | | | | | sequences |
| Wild type | AGGCTA | 58 | TAGATGC | 63 | |
| sequence (for | CGGCGC | | CTTTCTC | | |
| comparison) | TGTCAT | | TTGGAGC | | |
| | C | | TTA | | |
| TNF-α (-308G) | AGCCAA | 59 | CGTGAAC | 64 | 173 |
| | CCCCGT | | CCCGTCC | | |
| | TTTCT | | [symbol] | | |
| TNF-α (-308A) | AGCCAA | 60 | ATTTAAC | 65 | 178 |
| | CCCCGT | | TGAACCC | | |
| | TTTCT | | CGTCC [symbol] | | |
| Wild type | CTCCAA | 61 | TGGAGGC | 66 | |
| sequence (for | CCCCGT | | TGAACCC | | |
| comparison) | TTTCT | | CGTCC | | |
| | | | ( [symbol] / [symbol] ) | | |

Note:
Underlined: Tail sequence (5′ to 3′);
Bold: Target sequence or 3′ core sequence (5′ to 3′);
Bold/Italicized/Underlined: Mutation site or 3′ nucleotide.

TABLE 11
Reagent recipe for Multimodal Assay.

| Reagent | Final concentration |
|---|---|
| FASTSTART ™ Buffer (Without Mg) | 1X |
| (Hoffmann-La Roche; Basel, Switzerland) | |
| MgCl₂ | 1.7 mM |
| dNTPs | 0.2 mM each |
| Kras/Braf Primer Mix | 0.20 to 0.45 μM |
| PrimeraDx Universal Calibrators | 0.35X |
| FASTSTART ™ Taq (Hoffmann- | 0.9 U |
| La Roche; Basel, Switzerland) | |
| DNA/water | To a final volume of 25 μl |

TABLE 12
Thermocycling profile for Multimodal Assay.

| | Temperature | Duration | Number |
|---|---|---|---|
| Step | (° C.) | (sec) | of Cycles |
| Pre-heating | 96 | 360 | 1 |
| Amplification Phase 1 | 47/68/98 | 10/10/3 | 4 |
| | (anneal/extend/ | | |
| | strand separation) | | |
| Amplification Phase 2 | 53/72/98 | 5/5/3 | 17 |
| | (anneal/extend/ | | |
| | strand separation) | | |
| Amplification with | 53/72/98 | 5/220/3 | 19 |
| Sample Injection (for | (anneal/extend/ | | |
| Capillary Electropho- | strand separation) | | |
| resis Separation) | | | |

TABLE 13
Primer sequences for detection of single-nucleotide
point mutations in the KRAS and BRAF genes.

| | | SEQ |
|---|---|---|
| | | ID |
| | | NO: |
| BRAF Mutant & PCR Primers | | |
| Braf- | CCTATCGAATCAGTGGAAAAATAGC | 67 |
| 600-R4 | | |
| V600E- | CCGCATTTTGGTCTAGCTACAG [symbol] | 68 |
| AF2 | | |
| KRAS Mutant and PCR Primers | | |
| Kras- | CGCCTAGTGTATTAACCTTATGTGTG | 69 |
| 12-13- | | |
| F3 | | |
| G12R- | TCGCTGCCTACGCCAC [symbol] | 70 |
| CR2 | | |
| G12C- | CATTTGTTTGCCTACGCCAC [symbol] | 71 |
| TR | | |
| G12S- | CATTATACGAGTTGCCTACGCCAC [symbol] | 72 |
| AR | | |
| G12D- | CAATATCTAATGTCCTTGCCTACGCCA [symbol] | 73 |
| AR | | |
| G12A- | ACTATCGACGTCTTGCAACTTGCCTACGCCA [symbol] | 74 |
| CR | | |
| G12V- | CATATAACGGTCGAGGACTCTACTTGCCTACGCCA [symbol] | 75 |
| TR | | |
| G13R- | CTTCGACTTCAACGAAGTTGATGTCTCTTGCCTACGC [symbol] | 76 |
| CR | | |
| G13S- | CAATAATTAATTAACGTATAAATTCCCACTCTTGCCTACGC [symbol] | 77 |
| AR | | |
| G13C- | CTGTGACAACTTCATTGATCTGTTAAGTATTTCTCTTGCCTACGC [symbol] | 78 |
| TR3 | | |
| G13A- | CATCTCACTCATCATTACTAACTAGATAAACTTTAACTCTTGCCTACG [symbol] | 79 |
| CR3 | | |
| G13D- | CAAATTCTTTCATAAAATTCAATCTAAGTCATCTTTAACACTCTTGCCTACG [symbol] | 80 |
| AR5 | | |
| G13V- | CAATACTTTAGCTTGTCGATACTCAGTACAAATAGTGTTTAGCACTCTTGCCTACG [symbol] | 81 |
| TR2 | | |
| Wild-type KRAS Ref Gene Primers | | |
| Kras- | CTACACTTCATTAAGATTGGATCCAC | 82 |
| Ctrl-F1 | | |
| Kras- | CTCATTCCAAGTGTGTACTACTCCCAA | 83 |
| Ctrl-R1- | | |
| 204 | | |
| Kras- | CATTTGTTTCTTCAATCAAATGTTGATACTTACTTGGATGAAGTGTAAATATACTTTCTGA | 84 |
| Ctrl-R1- | AAACTAATAACGATATAATGAGCA | |
| 123 | | |

Note:
Unbold/Underlined: Tail sequence (5′ to 3′);
Bold: Target sequence (5′ to 3′);
Bold/Italicized/Underlined: Mutation site.

TABLE 14
Thermocycling conditions used in Example 4

| | Tempera- | | |
|---|---|---|---|
| # of cycles | ture (° C.) | Time | |
| | 96 | 360 | Sec |
| 4 cycles of: | 98 | 3 | sec |
| | 48 | 10 | sec |
| | 68 | 10 | sec |
| 17 cycles of: | 98 | 3 | sec |
| | 53 | 10 | sec |
| | 72 | 5 | sec |
| 19 cycles of: | 98 | 3 | sec |
| | 53 | 5 | sec |

| | |
|---|---|
| 72 | 224 sec (CE |
| | separation stage) |

TABLE 15

| | | SEQ ID |
|---|---|---|
| Name | Sequence | NO: |
| Kras-FAM- | 5′/56-FAM/ACT ATC GAC GTC TTG CAA CTT GCC TAC GCC AG-3′ | 163 |
| G12A-R | | |
| Kras-FAM- | 5′/56-FAM/CAT TTG TTT GCC TAC GCC ACA-3′ | 164 |
| G12C-R | | |
| Kras-FAM- | 5′/56-FAM/CAA TAT CTA ATG TCC TTG CCT ACG CCA T-3′ | 165 |
| G12D-R | | |
| Kras-FAM- | 5′/56-FAM/TCG TTG CCT ACG CCA CG-3′ | 166 |
| G12R-R | | |
| Kras-FAM- | 5′/56-FAM/CAT TAT ACG AGT TGC CTA CGC CAC T-3′ | 167 |
| G12S-R | | |
| Kras-FAM- | 5′/56-FAM/CAT ATA ACG GTC GAG GAC TCT ACT TGC CTA CGC CAA-3′ | 168 |
| G12V-R | | |
| Kras-FAM- | 5′/56-FAM/CTG TGA CAA CTT CAT TGA TCT GTT AAG TAT TTC TCT TGC CTA | 169 |
| G13C-R | CGC A-3′ | |
| Kras-FAM- | 5′/56-FAM/TTC TTT CAT AAA ATT CAA TCT AAG TCA TCT TTA ACA CTC TTG | 170 |
| G13D-R | CCT ACG T-3′ | |
| Kras-12- | 5′-CGC CTA GTG TAT TAA CCT TAT GTG TG-3′ | 171 |
| 13-F | | |
| Kras-FAM- | 5′/56-FAM/CAC CGA CAC AGC AGG TCA C-3′ | 172 |
| Q61H-C-F | | |
| Kras-FAM- | 5′/56-FAM/CAC CGA CAC AGC AGG TCA T-3′ | 173 |
| Q61H-T-F | | |
| Kras-FAM- | 5′/56-FAM/AGA TAG CAG GCT TGT AGA TCG ACA CAG CAG GTC T-3′ | 174 |
| Q61L-F | | |
| Kras-FAM- | 5′/56-FAM/CAA GAC TAG ATC ACT AGA CAC AGC AGG TCG-3′ | 175 |
| Q61R-F | | |
| Kras-Q61- | 5′-CTT TGA TAT TCA GTA GAC CTC TGT CAG CTT ATT ATA TTC AAT-3′ | 176 |
| R | | |
| Kras- | 5′-CAA AAT CTT GAA ACA ACC CTT TCC CAG AGA ACA AA-3′ | 177 |
| A146T-F | | |
| Kras-FAM- | 5′/56-FAM/CCA GCA TCG CAT TAC CTG TCT TGT CTT TGT-3′ | 178 |
| A146T-R | | |
| V600E- | 5′-CCGCATTTTGGTCTAGCTACAGAG-3′ | 179 |
| T/A-F | | |
| V600E- | 5′-CATACATAGATACATATAAATTTTGGTCTAGCTACAGAA-3′ | 180 |
| TG/AA-F | | |
| V600D- | 5′-GCATATCACATTTTGGTCTAGCTACAGAT-3′ | 181 |
| TG/AT-F | | |
| V600K- | 5′-CATCATGATCAATTGATTTTGGTCTAGCTACAAA-3′ | 182 |
| GT/AA-F | | |
| Braf-Tye- | 5′-/Tye665/ACCTATCGAAAATTTAATCAGTGGAAAAATAGC-3′ | 183 |
| V600-R | | |
| Kras- | 5′-CTC ATT CCA AGT GTG TAC TAC TCC CAA-3′ | 184 |
| Ctrl-R | | |
| Kras-Tye- | 5′-/Tye665/CTA CAC TTC ATT AAG ATT GGA TCC AC-3′ | 185 |
| Ctrl-F | | |
| G13D-1- | 5′/56-FAM/AA ATT CAA TCT AAG TCA TCT TTA ACA CTC TTG CCT ACG T-3′ | 186 |
| 28-2013 | | |

TABLE 16

| | | SEQ |
|---|---|---|
| | | ID |
| Name | Sequence | NO: |
| Kras-12- | 5′-CGC CTA GTG TAT TAA CCT TAT GTG TG-3′ | 187 |
| 13-F | | |
| Kras-A146T- | 5′-CAA AAT CTT GAA ACA ACC CTT TCC CAG AGA ACA AA-3′ | 188 |
| F | | |
| Kras-FAM- | 5′/56-FAM/CCA GCA TCG CAT TAC CTG TCT TGT CTT TGT-3′ | 189 |
| A146T-R | | |
| Kras-FAM- | 5′/56-FAM/ACT ATC GAC GTC TTG CAA CTT GCC TAC GCC AG-3′ | 190 |
| G12A-R | | |
| Kras-FAM- | 5′/56-FAM/CAT TTG TTT GCC TAC GCC ACA-3′ | 191 |
| G12C-R | | |
| Kras-FAM- | 5′/56-FAM/CAA TAT CTA ATG TCC TTG CCT ACG CCA T-3′ | 192 |
| G12D-R | | |
| Kras-FAM- | 5′/56-FAM/TCG TTG CCT ACG CCA CG-3′ | 193 |
| G12R-R | | |
| Kras-FAM- | 5′/56-FAM/CAT TAT ACG AGT TGC CTA CGC CAC T-3′ | 194 |
| G12S-R | | |
| Kras-FAM- | 5′/56-FAM/CAT ATA ACG GTC GAG GAC TCT ACT TGC CTA CGC CAA-3′ | 195 |
| G12V-R | | |
| Kras-FAM- | 5′/56-FAM/CTG TGA CAA CTT CAT TGA TCT GTT AAG TAT TTC TCT TGC CTA | 196 |
| G13C-R | CGC A-3′ | |
| Kras-FAM- | 5′/56-FAM/TTC TTT CAT AAA ATT CAA TCT AAG TCA TCT TTA ACA CTC TTG CCT | 197 |
| G13D-R | ACG T-3′ | |
| Kras-FAM- | 5′/56-FAM/CAC CGA CAC AGC AGG TCA C-3′ | 198 |
| Q61H-C-F | | |
| Kras-FAM- | 5′/56-FAM/CAC CGA CAC AGC AGG TCA T-3′ | 199 |
| Q61H-T-F | | |
| Kras-FAM- | 5′/56-FAM/AGA TAG CAG GCT TGT AGA TCG ACA CAG CAG GTC T-3′ | 200 |
| Q61L-F | | |
| Kras-FAM- | 5′/56-FAM/CAA GAC TAG ATC ACT AGA CAC AGC AGG TCG-3′ | 201 |
| Q61R-F | | |
| Kras-Q61-R | 5′-CTT TGA TAT TCA GTA GAC CTC TGT CAG CTT ATT ATA TTC AAT-3′ | 202 |
| V600E-T/ | 5′-CCGCATTTTGGTCTAGCTACAGAG-3′ | 203 |
| A-F | | |
| V600D- | 5′-GCATATCACATTTTGGTCTAGCTACAGAT-3′ | 204 |
| TG/AT-F | | |
| V600K- | 5′-CATCATGATCAATTGATTTTGGTCTAGCTACAAA-3′ | 205 |
| GT/AA-F | | |
| V600E- | 5′-CATACATAGATACATATAAATTTTGGTCTAGCTACAGAA-3′ | 206 |
| TG/AA-F | | |
| Braf-Q670- | 5′-/Q670/ACCTATCGAAAATTTAATCAGTGGAAAAATAGC-3′ | 207 |
| V600-R | | |
| Kras-Q670- | 5′-/Q670/CTA CAC TTC ATT AAG ATT GGA TCC AC-3' | 208 |
| Ctrl-F | | |
| Kras-Ctrl-R | 5′-CTC ATT CCA AGT GTG TAC TAC TCC CAA-3′ | 209 |

1-110. (canceled)

111. A method for determining the presence of one or more target polynucleotides, the method comprising;
performing a PCR amplification regimen comprising cycles of strand separation, primer annealing, and primer extension on a reaction mixture comprising a nucleic acid sample and a set of oligonucleotide primers specific for each target polynucleotide;
wherein each said set of oligonucleotide primers comprises a first subset of at least one dual domain forward primer and a second subset of at least one reverse primer;
wherein each dual domain primer of a set comprises a 5′ tail region that differs from the 5′ tail region of other dual domain primers in the set and a 3′ core region complementary to a sequence on one strand of a double-stranded nucleic acid comprising said target;
wherein for each dual domain primer, the 3′ core substantially anneals to its complementary target site sequence at a first annealing temperature, and the sequence comprised by the 5′ tail and 3′ core region substantially anneals to its complement at a second annealing temperature, the second annealing temperature being higher than the first annealing temperature, such that at said second annealing temperature said 3′ core of said dual domain primer cannot substantially anneal to a template molecule that does not also have the complement of the dual domain primer's 5′ tail sequence;
wherein for each dual domain primer of a primer set:
the 5′ tail sequence does not have any homology to the target sequence; and
the 5′ tail sequence has 6 or fewer contiguous homologous bases relative to any other 5′ tail sequence of the dual domain primer set;
wherein said PCR amplification regimen comprises first and second phases,
the first phase comprising annealing at said first annealing temperature for a first set of cycles, and;
the second phase comprising annealing at said second annealing temperature for a second set of cycles;
and detecting an amplified product for each target polynucleotide, wherein said detecting indicates the presence of said target polynucleotide.

112. The method of claim 111, wherein each dual domain primer of a set comprises a 5′ tail region that differs from the 5′ tail region of other dual domain primers in the set, a 3′ core region complementary to a sequence on one strand of a double-stranded nucleic acid comprising said target, and a terminal nucleotide complementary to one of the variant nucleotides occurring at said target site.

113. The method of claim 111, wherein at least one set of primers comprises a truncated dual domain forward primer.

114. The method of claim 111, wherein a reverse primer is selected from the group consisting of:
an amplifying primer; a dual domain primer; and a truncated dual domain primer.

115. The method of claim 111, wherein said target polynucleotides are selected from the group consisting of:
polynucleotides comprising SNPs; polynucleotides comprising alleles of SNPs;
protein coding DNA or RNA polynucleotides; non-protein coding DNA or RNA polynucleotides; polynucleotides comprising mutations; polynucleotides comprising deletions; and polynucleotides comprising insertions.

116. The method of claim 111, wherein a plurality of target polynucleotides are detected in a single reaction.

117. The method of claim 116, wherein the target polynucleotides comprise at least one polynucleotide selected from the group consisting of:
polynucleotides comprising SNPs and polynucleotides comprising alleles of SNPs;
and at least one polynucleotide selected from the group consisting of:
protein coding DNA or RNA polynucleotides; non-protein coding DNA or RNA polynucleotides; polynucleotides comprising mutations; polynucleotides comprising deletions; and polynucleotides comprising insertions.

118. The method of claim 111, wherein at least one of the target polynucleotides comprises a SNP and wherein said method genotypes the nucleotide at said SNP.

119. The method of claim 118, wherein dual domain or truncated dual domain primers specific for each of 2 or more alleles of a target SNP are present in the same reaction.

120. The method of claim 118, wherein one dual domain or truncated dual domain primer of a set is specific for the predominant allele of the target SNP.

121. The method of claim 116, wherein the dual domain or truncated dual domain primers specific for each of 2 or more alleles or sequence variants of a target nucleotide are present in the same reaction.

122. The method of claim 121, wherein one dual domain or truncated dual domain primer of a set is specific for the predominant allele or variant allele of the target polynucleotide.

123. The method of claim 111, wherein the amplified products of two or more members of a primer subset can be distinguished.

124. The method of claim 123, wherein the products of two or more members of a primer subset are distinguished by a method selected from the group consisting of:
being of distinct sizes; being labeled with different detectable labels; sequencing the amplified products; oligonucleotide hybridization; and melting-curve analysis of the amplified products.

125. The method of claim 111, wherein the core sequence is 10 to 40 nucleotides in length.

126. The method of claim 111, wherein the first phase comprises from 1 to 10 cycles.

127. The method of claim 111, wherein each 5′ tail sequence of a set has fewer than 3 contiguous homologous bases relative to the other 5′ tail sequences of the set.

128. The method of claim 111, wherein the 5′ tail sequence is from 1 to 200 nucleotides in length.

129. The method of claim 111, wherein the second annealing temperature is at least 4° C. higher than the first annealing temperature.

130. The method of claim 111, wherein the dual domain or truncated dual domain primers present in a reaction have Tₘ values for their respective 3′ core regions that vary by no more than 8° C. from each other.
